(19) United States
(12) Patent Application Publication
(10) Pub. No.: US 2017/0211156 A1
(43) Pub. Date: Jul. 27, 2017

(54) Quantitative Reverse Transcription Polymerase Chain Reaction Kit for Breast Cancer Drug Screening Test and Early Diagnosis Using Tissue and Blood

(71) Applicants: Optipharm Co., Ltd., Cheongju-si (KR); University Industry Foundation, Yonsei University Wonju Campus, Wonju-si (KR)

(72) Inventors: Hye Young Lee, Wonju-si (KR); Seung Il Kim, Seoul (KR); Hye Young Wang, Gunsan-si (KR); Sang Jung Park, Asan-si (KR)

(73) Assignees: Optipharm Co., Ltd., Cheongju-si (KR); University Industry Foundation, Yonsei University Wonju Campus, Wonju-si (KR)

(21) Appl. No.: 15/454,914

(22) Filed: Mar. 9, 2017

Related U.S. Application Data
(63) Continuation-in-part of application No. 14/241,365, filed on Feb. 26, 2014, now abandoned.

(30) Foreign Application Priority Data
Sep. 24, 2013 (KR) 1020130113497

(51) Int. Cl. C12Q 1/68

(52) U.S. Cl. CPC C12Q 1/6886; C12Q 2600/158; C12Q 2600/112

(57) ABSTRACT

There are provided an information offering method for diagnosing breast cancer comprising: (a) separating total RNA from a cell obtained from a tissue or blood of a suspected cancer patient; (b) synthesizing a cDNA from the separated total RNA; (c) performing a real-time PCR with the synthesized cDNA by using primer pair mix and probe mix that can amplify human epidermal growth factor receptor 2 (HER2); wherein, the primer pair mix that can amplify human epidermal growth factor receptor 2 (HER2) are described as SEQ ID Nos.:1 and 2, 3 and 4, and 6 and 7 and the probe mix are described as SEQ ID Nos.: 5, 8, and 9.

### RELATED PATENT DATA

This application is a continuation-in-part of U.S. patent application Ser. No. 14/241,365 filed Feb. 26, 2014 which is a 35 U.S.C. §371 application and claims priority to International Application No. PCT/KR2013/008589, filed on Sep. 25, 2013, which claims priority to KR 10-2013-0113497, filed on Sep. 24, 2013, the entirety of each of which is hereby incorporated by reference.

### SEQUENCE LISTING

The present application includes a sequence listed the text form of which is included as an appendix to the application. Incorporated by reference herein is the material in the 5.62 KB (5,758 bytes) .txt file submitted in computer readable form (CRF), created Jun. 22, 2015 entitled HA134-002_ST25.txt.

### TECHNICAL FIELD

The present invention relates to a method for a breast cancer drug screening test and early diagnosis using tissues and blood and a quantitative reverse transcription polymerase chain reaction kit thereof.

### BACKGROUND ART

Human epidermal growth factor receptor 2 (HER2) is a member of the ErbB-like oncogene family, and a HER2 test is very important in treating breast cancer (Di Fiore P P, et al. (1987) Cell 51(6): 1063-1070.). In particular, HER2-overexpression can be seen from 20 to 30% of breast cancer patients, and such HER2-overexpressing patients have poor prognoses and develop severe breast cancer, whereby their survival time is reduced by 5 years as compared with other patients without HER2-overexpression (Revillion F, Lhotellier V, Hornez L, Bonneterre J, & Peyrat J P (2008) Ann Oncol 19(1): 73-80.). In order to treat such HER2-overexpressing patients, Herceptin (Roche) has been used. Herceptin is a humanized monoclonal antibody and directly targets an extracellular domain of a HER2 receptor. Currently, Herceptin has been used for chemical therapy in treating metastatic breast cancer patients and neoadjuvant patients (Piccart-Gebhart M J (2006) Eur J Cancer 42(12): 1715-1719.).

As the best gold standard for determining a stage and a prognosis of cancer or screening a drug in diagnosing a cancer patient, a fluorescence in situ hybridization (FISH) method and an immunohistochemical staining (IHC) method have been used. In particular, in diagnosing HER2-overexpression, the IHC method has been used to show whether or not there is overexpression of a HER2 protein on a cancer cell surface, and the FISH method has been used to check whether or not there is overexpression of a HER2 gene on a chromosome (Sauerbrei W, et al. (2000) J Clin Oncol 18(1): 94-101.). In particular, the IHC method is the most widely used method as a primary screening test. However, the IHC method is different in each test institute and has been a controversial matter in terms of technical accuracy or reproducibility of result. Further, it is known that the FISH method has a higher concordance rate of result than the IHC method has improved sensitivity and specificity as compared with the IHC method.

However, it is known that since the FISH method has a very complicated test process and uses fluorescence, a result of the FISH method cannot be perpetuated.

Furthermore, it is known that since a fluorescent probe is very expensive, the FISH method cannot be carried out in small-scale hospitals or the like (Lewis F, et al. (2004) Histopathology 45: 207-17).

As relevant prior art documents, there are Korean Patent Laid-open Publication No. 10-2009-0079845 relating to “Protein markers for monitoring, diagnosis, and screening of breast cancer and the method of monitoring, diagnosis, and screening using thereof” and Korean Patent Laid-open Publication No. 10-2009-0064378 relating to “Genes and polypeptides relating to breast cancers”.

### DISCLOSURE

### Technical Problem

The present invention is contrived in order to solve the conventional problems and satisfy the need, and an object of the present invention is to provide an information offering method for diagnosing breast cancer by using a quantitative reverse transcription polymerase chain reaction.

Another object of the present invention is to provide a kit for diagnosing breast cancer.

### Technical Solution

In order to achieve the above objects, an exemplary embodiment of the present invention provides an information offering method for diagnosing breast cancer comprising: (a) separating total RNA from a cell obtained from a tissue or blood of a suspected cancer patient; (b) synthesizing a cDNA from the separated total RNA; (c) performing a real-time PCR with the synthesized cDNA by using primer pair mix and probe mix that can amplify human epidermal growth factor receptor 2 (HER2);

wherein, the primer pair mix that can amplify human epidermal growth factor receptor 2 (HER2) are described as SEQ ID Nos.:1 and 2, 3 and 4, and 6 and 7 and the probe mix are described as SEQ ID Nos.: 5, 8, and 9.

Another exemplary embodiment of the present invention provides an information offering method for diagnosing breast cancer comprising: (a) separating a full-length RNA from a cell obtained from a tissue or blood of a suspected cancer patient; (b) synthesizing a cDNA from the separated full-length RNA; (c) performing a realtime PCR with the synthesized cDNA by using at least one primer pair and probe selected from the group consisting of a primer pair and a probe that can amplify human epidermal growth factor receptor 2 (HER2), a primer pair and a probe that can amplify cytokeratin 19, a primer pair and a probe that can amplify epithelial cell adhesion molecule (EpCAM), a primer pair and a probe that can amplify a human telomerase reverse transcriptase (hTERT), a primer pair and a probe that can amplify Ki67, and a primer pair and a probe that can amplify vimentin, and a primer pair and a probe that can amplify a glyceraldehyde-3-phosphate dehydrogenase (GAPDH); and (d) comparing an amount of the amplification with an expression amount to a normal person.

A method for separating a full-length RNA (Total RNA) and a method for synthesizing a cDNA from the separated full-length RNA that are generally used can be performed through the publicly known method, and the detailed description about the process is disclosed in Joseph Sambrook et al., Molecular Cloning, A Laboratory Manual, Cold Spring Harbor Laboratory Press, Cold Spring Harbor, N.Y. (2001); and Noonan, K. F., and the like, which may be incorporated in the present invention as a reference.

The primer of the present invention can be chemically synthesized using a phosphoramidite solid support method, or other publicly known methods. The nucleic acid sequence may be also deformed by using many ways known in the prior art. Nonlimited examples of such deformation may include methylation, “a capping,” substitution to analogues of at least one natural nucleotide, and deformation between nucleotides, for example, deformation into an uncharged connector (for example, methyl phosphonate, phosphotriester, phosphoramidate, carbamate, or the like) or a charged connector (for example, phosphorothioate, phosphorodithioate, or the like).

Nucleic acid may contain at least one additional covalently bonded residue, for example, proteins (for example, nuclease, toxin, antibody, signal peptide, poly-L-lysine, or the like), an intercalator (for example, acridine, psoralene, or the like), a chelating agent (for example, a metal, a radioactive metal, iron, an oxidative metal, or the like), and an alkylating agent. The nucleic acid sequence of the present invention may also be deformed by using a marker capable of directly or indirectly providing a detectable signal. For example, the marker may include a radioactive isotope, a fluorescent molecule, biotin, and the like.

In the method according to the present invention, the amplified target sequence (HER2 and GAPDH genes) may be marked with a detectable marker material. In an exemplary embodiment, the marker material may be, but not limited to, a material that emits fluorescence, phosphorescence, chemiluminescence, or radioactivity. Preferably, the marker material may be fluorescein, phycoerythrin, rhodamine, and lissamine Cy-5 or Cy-3. The marker material may be marked with a detectable fluorescent marker material by performing an RT-PCR through marking Cy-5 or Cy-3 to a 5′-terminal and/or a 3′-terminal of a primer when amplifying a target sequence.

Further, the marking using the radioactive material may be achieved by marking an amplified product with radioactivity through incorporating the radioactivity to the amplified product while synthesizing the amplified product by adding a radioactive isotope, such as ³²P or ³⁵S to a PCR reaction solution when performing the RT-PCR. At least one oligonucleotide primer set used for amplifying the target sequence may be used.

The marking may be performed through various methods typically performed in the art, such as, a nick translation method, a random priming method [Multiprime DNA labelling systems booklet, “Amersham” (1989)], and a kination method [Maxam & Gilbert, Methods in Enzymology, 65:499 (1986)]. The marker provides a signal which can be detected by fluorescence, radioactivity, chromophore measurement, weight measurement, X-ray diffraction or absorption, magnetism, enzymatic activity, amass analysis, binding affinity, hybridization high frequency, and a nano crystal.

According to one aspect of the present invention, in the present invention, an expression level is measured in the level of mRNA through an RT-PCR. To do so, a new primer pair and a fluorescent-marked probe that are specifically bonded to the HER2 and GAPDH genes are required. The primer and probe specified with the specific base sequence in the present invention can be used, but the present invention is not limited thereto. As long as they provide a detectable signal by specifically binding to these genes and can perform an RT-PCR, they can be used without limitation. In the above descriptions, FAM and Quen (Quencher) mean a fluorescent dye.

The RT-PCR method applied to the present invention can be performed through the known process typically used in the art.

The measurement of the mRNA expression level can be used without limitation as long as it can measure a general mRNA expression level, and depending on a type of a probe marker used, it can be performed through, but not limited to, radioactivity measurement, fluorescence measurement, or phosphorescence measurement. As one of methods for detecting an amplified product, a fluorescence measurement method can be performed by marking Cy-5 or Cy-3 to a 5′-terminal of a primer; performing a realtime RT-PCR with the marked primer to mark a detectable fluorescent marker material to a target sequence; and measuring the marked fluorescence by using a fluorometer. In addition, a radioactivity measurement method can be performed by marking an amplified product with a radioactive isotope such as ³²P or ³⁵S, and the like by adding the radioactive isotope to a PCR reaction solution when performing an RT-PCR; and measuring radioactivity by using a radioactivity measurement instrument, such as a Geiger counter or a Liquid Scintillation Counter.

According to an exemplary embodiment of the present invention, a fluorescent-marked probe is attached to a PCR product amplified through the PCR to fluoresce at a specific wavelength; the expression levels of mRNAs of the genes of the present invention are measured in real time with a fluorometer of a PCR apparatus at the same time when the amplification is carried out; and then the measured values are calculated and visualized through a PC so that a tester can easily confirm the expression levels.

According to another aspect of the present invention, the diagnostic kit may be a kit for diagnosing breast cancer that includes essential components required for performing a reverse transcription polymerase chain reaction. The reverse transcription polymerase chain reaction kit may include primer pairs respectively specific to the genes of the present invention. The primer may be a nucleotide having a specific sequence, in a range of from about 7 bp to about 50 bp and more preferably from about 10 bp to about 30 bp, at a nucleic acid sequence of each marker gene.

In addition, thereto, the reverse transcription polymerase chain reaction kit may include a test tube or other appropriate container, a reaction buffer solution (various in pH and magnesium concentration), deoxynucleotides (dNTPs), a Taq-polymerase, and an enzyme such as a reverse transcriptase, a DNAse, an RNAse inhibitor, DEPC-water, sterilized water, etc.

The term, “information offering method for diagnosing cancer” in the present invention is a preliminary stage for diagnosis to offer objective basic information required for diagnosing cancer, but does not include a clinical judgment and opinion by a doctor.

The term, “primer” indicates a nucleic acid sequence having a short free 3′-terminal hydroxyl group; can form a complementary template and base pair; and indicates a short nucleic acid sequence acting as a starting point for template strand transcription. The primer can start DNA synthesis under presence of different four nucleoside triphosphates and a reagent for a polymerization (that is, DNA polymerase or reverse transcriptase) in a proper buffer solution and at a proper temperature. The primer according to the present invention as a primer specific to each marker gene is sense and anti-sense nucleic acid having 7 to 50 nucleotide sequences. The primer may be incorporated with an additional feature which cannot change a basic property of a primer that acts as a starting point of DNA synthesis.

The term, “probe” is a single chain nucleic acid molecule, and includes a sequence complementary to a target nucleic acid sequence.

The term, “realtime reverse transcription polymerase chain reaction (realtime RT-PCR)” is a molecular biological polymerization method in which a cDNA is produced after reverse transcription of an RNA with a complementary DNA (cDNA) using a reverse transcriptase, and then a target is amplified using a target probe including a target primer and a marker using the cDNA as a template at the same time when a signal generated at the marker of the target probe is quantitatively detected.

In an exemplary embodiment of the present invention, preferably, the comparing an amount of the amplification with an expression amount to a normal person is carried out by a standard or Cut-Off value. In the above-described method, preferably, a Ct value of the GAPDH is set to 30 or less, and the Ct value refers to the number of a cycle where amplification starts to remarkably increase during a PCR process, but it is not limited thereto.

In another exemplary embodiment of the present invention, preferably, the primer that can amplify human epidermal growth factor receptor 2 (HER2) is described as Sequence Nos. 1 to 2, 3 to 4, or 6 to 7 and the probe is described as Sequence No. 5,8, or 9; the primer pair and the probe that can amplify cytokeratin 19 are described as Sequence Nos. 13 to 14, and 15, respectively; the primer pair and the probe that can amplify epithelial cell adhesion molecule (EpCAM) are described as Sequence Nos. 16 to 17, and 18, respectively; the primer pair and the probe that can amplify a human telomerase reverse transcriptase (hTERT) are described as Sequence Nos. 19 to 20, and 21, respectively; the primer pair and the probe that can amplify Ki67 are described as Sequence Nos. 22 to 23, and 24, respectively; the primer pair and the probe that can amplify vimentin are described as Sequence Nos. 25 to 26, and 27, respectively, but they are not limited thereto.

In still another exemplary embodiment of the present invention, preferably, the primer pair that can amplify a GAPDH is described as Sequence Nos. 10 and 11 and the probe has a base sequence described as Sequence No. 12, but they are not limited thereto.

Further, an exemplary embodiment of the present invention provides a primer pair and a probe for diagnosing breast cancer comprising: at least one primer pair and probe selected from the group consisting of a primer pair described as Sequence Nos. 1 to 2, 3 to 4, or 6 to 7 and a probe is described as Sequence No. 5, 8, or 9 that can amplify human epidermal growth factor receptor 2 (HER2), a primer pair described as Sequence Nos. 13 to 14 and a probe described as Sequence No. 15 that can amplify cytokeratin 19, a primer pair described as Sequence Nos. 16 to 17 and a probe described as Sequence No. 18 that can amplify epithelial cell adhesion molecule (EpCAM), a primer pair described as Sequence Nos. 19 to 20 and a probe described as Sequence No.21 that can amplify a human telomerase reverse transcriptase (hTERT), a primer pair described as Sequence Nos. 22 to 23 and a probe described as Sequence No. 24 that can amplify Ki67, and a primer pair described as Sequence Nos. 25 to 26 and a probe described as Sequence No. 27 that can amplify vimentin; and a primer pair described as Sequence Nos. 10 and 11 and a probe described as Sequence No. 12.

In an exemplary embodiment of the present invention, preferably, a 5′-terminal of the probe is marked with a fluorescent material, but it is not limited thereto.

Furthermore, an exemplary embodiment of the present invention provides a composition for diagnosing breast cancer comprising: the primer pair and the probe of the present invention.

Moreover, an exemplary embodiment of the present invention provides a kit for diagnosing breast cancer comprising the composition of the present invention.

Besides, an exemplary embodiment of the present invention provides a kit for early diagnosing breast cancer or diagnosing breast cancer by stage comprising the composition of the present invention.

Hereinafter, the present invention will be explained.

In order to substitute for this, based on an RT-qPCR method capable of simply and quantitatively producing a result, an expression amount obtained after an HER2 gene using a GAPDH as a reference gene is amplified is comparatively quantitated and an expression rate is compared with results of the conventional methods, IHC and FISH.

Further, the present invention is completed for more effective treatment for and diagnosis of breast cancer with expression levels of HER2 expressed in blood and a cancer-related marker in blood as well as a tissue sample.

### Effect

As can be seen from the present invention, the present invention can be helpful for more effective treatment for and diagnosis of breast cancer with expression levels of HER2 expressed in blood and a cancer-related marker in blood as well as a tissue sample.

### DESCRIPTION OF DRAWINGS

FIG. 1 is provided to check HER2 sensitivity of an RT-qPCR using a SK-BR-3 cell line;

FIG. 2 is provided to compare expression levels of HER2 mRNA using a breast cancer cell line;

FIG. 3 is provided to show a result of analyzing correlation between HER2 RT-qPCR and HER2 IHC;

FIG. 4A-C show an ROC curve analysis method for determining clinical Cut-Off values;

FIG. 5 shows clinical Cut-Off values set by using an ROC curve analysis method;

FIG. 6 shows results of clinical specimens using an RT-qPCR;

FIG. 7 is provided to check sensitivity of an HER2 RT-qPCR after a SK-BR-3 is mixed into blood of a normal person;

FIG. 8 is provided to compare expression of HER2 in blood of a normal person group and a breast cancer patient group;

FIG. 9A-D are provided to compare expression levels of cancer-related markers in blood;

FIG. 10A-D are provided to compare whether or not a cancer-related marker is expressed in blood of a normal person and a breast cancer patient;

FIG. 11 is provided to compare an expression level of a cancer-related marker in blood and an expression level of an HER2 in blood using an RT-qPCR;

FIG. 12 is provided to analyze correlation between expression of an HER2 in blood and expression levels of Ki67 and hTER;

FIG. 13 is provided to analyze correlation between histological grade and hTERT and Ki67;

FIG. 14 is provided to compare histological expression of an HER2 and an expression level of an HER2 in blood;

FIG. 15 is provided to check an expression level of an epithelial antigen of a breast cancer patient by stage;

FIG. 16A-B are provided to check an expression level of a cancer-related marker in a cell by stage of breast cancer;

FIGS. 17-20 are the result of an RT-qPCR using single primer pair & probe (SEQ.ID No.1-2, and 5 in FIG. 17; SEQ.ID No.3-4, and 5 in FIG. 18; SEQ.ID No.6-7, and 8 in FIG. 19; or SEQ.ID No.6-7, and 9 in FIG. 20, respectively); and

FIGS. 21-24 are the multiplex one-tube nested PCR result of an RT-qPCR using primer pairs & probes (SEQ.ID No.1-4, and 5 in FIGS. 21-22; SEQ.ID No.1-9 in FIGS. 23-24, respectively), specially, in FIGS. 23-24, all primers and probes set forth in SEQ. ID 1-9 are used in the PCR.

### BEST MODE

Hereinafter, examples of the present invention will be described in detail with reference to the accompanying drawings. The following examples are provided only for illustrating the present invention. Therefore, it is obvious to an ordinary skilled person in the art that the scope of the present invention should not be construed as being limited by the following examples according to the gist of the present invention.

### EXAMPLE 1

### Biopsy Method for Drug Screening Test

(1) Materials

There were used FFPE (Formalin Fixed Paraffin Embedded) tissues of 199 patients in the Shinchon Severance Hospital from 2010 to 2011. Whether or not there was histological expression of an HER2 of a patient was checked by performing an immunohistochemical staining (IHC) method and a fluorescence in situ hybridization (FISH) method. Further, in order to check whether or not there was expression of an HER2, breast cancer cell lines such as SK-BR3, MCF7, and MDA-MB 231 were used.

(2) Immunohistochemical Staining (Immunohistochemistry; IHC) Method

A paraffin block was sectioned to a thickness of 4 μm and then attached to a slide and dried sufficiently. Then, immunohistochemical staining was carried out by using an automatic immunohistochemical staining apparatus, BenchMark ST (Ventana medical system, USA). As a primary antibody, polyclonal rabbit anti-human c-erbB-2 oncoprotein (A0485, DakoCytomation, Glostrup, Denmark) was used as being diluted at 1:1000. The slide was stained in this way, and then expression of the HER2 protein was evaluated into 4 grades, i.e. 0, 1+, 2+, and 3+, depending on a staining degree of the HER2 protein at a cell membrane of the cancer cell. In the case of 0 or 1+, it was diagnosed as HER2 negative, and in the case of 3+, it was diagnosed as HER2 positive.

In the case of 2+, it was diagnosed as HER2 positive or by performing the FISH depending on clinical information of a patient.

(3) Fluorescence In Situ Hybridization (FISH) Method

With respect to the patients evaluated as 2+ according to the HER2 IHC method, a tissue block fixed by paraffin was sectioned to a thickness of 4 μm by using a microtome and attached to a slide, and underwent a deparaffinization and rehydration to be used for an experiment conducted by using a commercialized HER2 DNA probe kit (Vysis Inc, Downers Grove, Ill., USA) according to the protocol of the manufacturer.

Whether or not there was expression of an HER2 was evaluated depending on an expression level of a gene, and when an amplification index was 2.2 or more, it was determined as HER2 positive.

(4) Separation of Total RNA from Separated Tissue

Two segments sectioned from the FFPE tissue to a thickness of 10 μm underwent deparaffinization, and RNAs were extracted by using an automatic nucleic acid extraction apparatus MagNApure LC RNA Isolation Kit III (Roche).

The cell number of each cell line was set to 1×10⁶ and a total RNA was separated by using trizol according to the protocol of the manufacturer. The separated total RNA was quantitated by using a NanoQuant system (TECAN).

(5) Production of cDNA from Separated Total RNA and Performance of Realtime PCR

a. Synthesis of cDNA

cDNA was synthesized by adding 0.5 to 3 μg of the separated total RNA, 0.25 μg of a random primer (Invitrogen), 250 μM of dNTP (Intron), 50 mM Tris-HCl (pH 8.3),75 mM KCl, 3 mM MgCl₂, 8 mM DTT, and 200 units of MMLV reverse transcription polymerase (Invitrogen), further adding DW treated with DEPC to be a final volume of 30 μl, mixing, and then reacting the synthesizing reaction solution at 25° C. for 10 minutes, at 37° C. for 50 minutes, and then at 70° C. for 15 minutes in a thermocycler (ABI).

b. Performance of RT-qPCR

For a composition of a realtime PCR reactant, 25 mM TAPS (pH 9.3 at 25° C),50 mM KCl, 2 mM MgCl₂, 1 mM 2-mercaptoethanol, 200 μM each dNTP, 1 unit of a Taq polymerase (TAKARA), 10 pmole of a Forward primer, 10 pmole of a Reverse primer, 10 pmole of a probe, and 2 μl of the synthesized cDNA were added, and a realtime PCR was carried out to be a final volume of 20 μl. The primers and probes had the following base sequences, respectively.

Primer and Probe for HER2

Forward (1):
(Sequence No. 1)
5′AACCTGGAACTCACCTACCTGCCCAC-3
Reverse (1):
(Sequence No. 2)
5′CGATGAGCACGTAGCCCTGCAC-3
Forward (2):
(Sequence No. 3)
5′AACTCACCTACCTGCCCACCAAT-3
Reverse (2):
(Sequence No. 4)
5′CACGTAGCCCTGCACCTCCT-3
Probe (1):
(Sequence No. 5)
5′FAM-CAGCCTGTCCTTCCTGCAGGATATC-BHQ1-3
Forward (3):
(Sequence No. 6)
5′AAGCATACGTGATGGCTGGTGT-3
Reverse (3):
(Sequence No. 7)
5′TCTAAGAGGCAGCCATAGGGCATA-3
Probe (2):
(Sequence No. 8)
5′FAM-ATATGTCTCCCGCCTTCTGGGCATCT-BHQ1-3
Probe (3):
(Sequence No. 9)
5′FAM-CATCCACGGTGCAGCTGGTGACACA-BHQ1-3

Primer and Probe for GAPDH

Forward:
(Sequence No. 10)
5′CCATCTTCCAGGAGCGAGATCC-3
Reverse:
(Sequence No. 11)
5′ATGGTGGTGAAGACGCCAGTG-3
Probe:
(Sequence No. 12)
5′FAM-TCCACGACGTACTCAGCGCCAGCA-BHQ1-3

The PCR was carried out by using an ABI 7500Fast (Applied Biosystem) one time at a denaturation temperature of 94° C. for 5 minutes; a cycle of a denaturation temperature of 95° C. for 30 seconds and an annealing temperature of 60° C. was first carried out 10 times; and a cycle of a denaturation temperature of 95° C. for 30 seconds and an annealing temperature of 55° C. for 40 seconds was repeatedly carried out 40 times. In addition, a fluorescence measurement process was added after each of the annealing processes to measure a fluorescent value that was increased in each cycle.

(6) Result Analysis

Each of experiment results was analyzed by using 7500 Software v2.0.4 (Applied Biosystem). SK-BR3 as a breast cancer cell was diluted by stages from 10⁵ to 1 cell, a relative quantitative curve was drawn, and expression amounts were comparatively quantitated by using Ct values to check expression rates. In this case,

each of HER2 expression amounts was compared based on an expression amount of GAPDH, and after an HER2 expression amount of MDA-MB-231 as a HER2 negative breast cancer cell line was set to 1 as a reference value, an HER2 expression amount of each specimen and cell line was presented.

(7) Check of Amplification Through Software Analysis and Quantitation of Amplified Product

By using a comparative Ct method as one of methods for quantitating an expression amount of a gene of qRT-PCR, measurement was made according to the following relation function. This function was set in ABI 7500 software-Bio-Rad CFX Manager Software, and, thus, automatic calculation was made and a result was output.

[Relation Function 1]

ΔΔCt=ΔCt(sample)−ΔCt(reference gene)

Herein, the Ct value refers to the number of a cycle where amplification starts to remarkably increase during a PCR process.

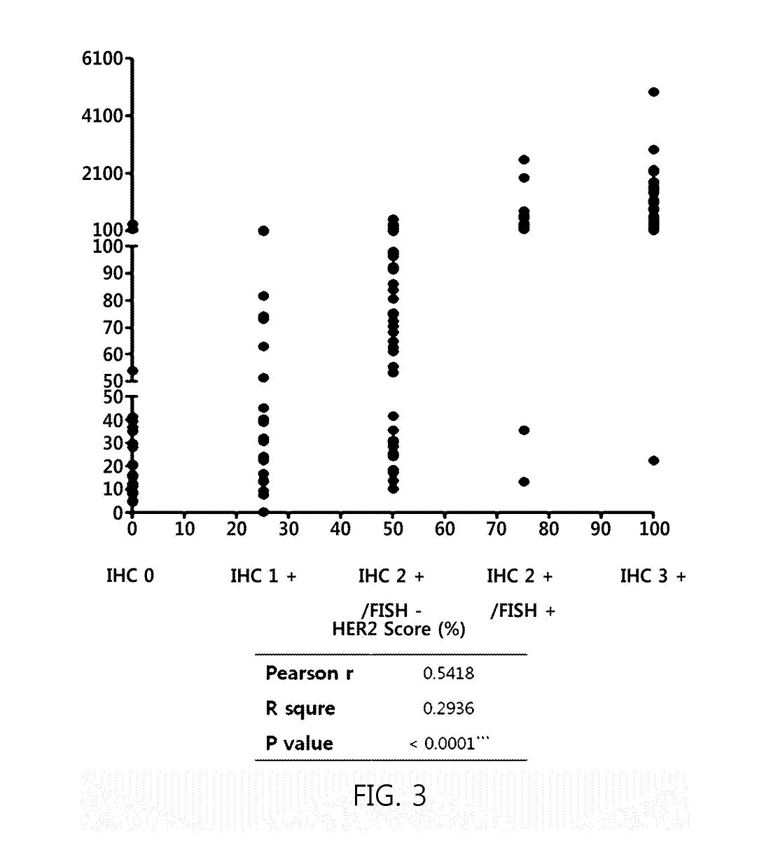

ΔΔCt means a value (mRAN expression ratio) of the vertical axis in FIG. 3.

[Relation Function 2]

Relation function for analyzing HER2 expression amount in positive control group

ΔCt value of SKBR3=Ct value of HER2 in SKBR3−Ct value of reference gene (GAPDH) in SKBR3

ΔCt value of THP-1=Ct value of HER2 in THP-1−Ct value of reference gene (GAPDH) in THP-1

R(expression amount)=ΔCt value of SKBR3−ΔCt value of THP-1

[Relation Function]

Relation function for analyzing HER2 expression amount in tissue sample of breast cancer patient

ΔCt value in tissue of breast cancer patient=Ct value of HER2 in tissue of breast cancer patient−Ct value of reference gene (GAPDH) in tissue

ΔCt value of THP-1=Ct value of HER2 in THP-1−Ct value of reference gene (GAPDH) in THP-1

R(expression amount)=ΔCt value in tissue of breast cancer patient−ΔCt value of THP-1

A Ct value of the reference gene used in the present experiment refers to a Ct value of GAPDH, and the reference gene may include other house-keeping genes in addition to the GAPDH used in the present experiment.

SKBR3: serving as a positive control and used for checking whether HER2 is actually overexpressed or not.

### EXAMPLE 2

### Analysis of Cancer Expression Marker in Blood

(1) Specimens

There was used blood offered by 188 breast cancer patients and donated by 50 normal persons without breast cancer to the Yonsei University Severance Hospital from 2011 to 2012.

(2) Separation of Cell from Patient's Blood

Blood was collected from a vein of a cancer patient and a vein of a normal person into 2 tubes with EDTA anticoagulant. In order to prevent contamination from epithelial cells, 5 ml of the first collected blood was removed and 10 ml of the later collected blood was used in the present test. In order to prevent damage to mRNA in the patient's blood, an erythrolysis treatment as a first process of the experiment was started within 4 hours of the blood collection. In order to dissolve erythrocytes from the blood, an erythrocyte dissolving solution including 154 mM NH₄Cl, 9 mM KHCO₃, and 0.1 mM EDTA was added in a volume five times, vortexed, stagnated at room temperature for 10 minutes, and centrifuged at 600 g at 4° C. for 10 minutes. A supernatant was removed carefully. In order to remove the remaining erythrocytes, 10 ml of an RBC lysis buffer was added and stagnated in ice for 5 minutes and centrifuged again at 3000 rpm for 2 minutes at 4° C. A supernatant was removed carefully, and then, 1 ml of PBS was added to resuspend a pellet and then treated with an RNase A (100 μg/ml) for 5 minutes to remove free nucleic acids present in the blood.

(3) Separation of Total RNA from Separated Cell

The resuspended pellet was centrifuged again at 3000 rpm for 2 minutes at 4° C., and a supernatant was removed by pipetting. Then, 1 ml of a trizol agent (Invitrogen) was added and a total RNA was separated according to the protocol of the manufacturer.

(4) Production of cDNA from Separated Total RNA and Performance of Realtime PCR

a. Synthesis of cDNA

cDNA was synthesized by adding 2 μg of the separated total RNA, 0.25 μg of a random hexamer (Invitrogen), 250 μM of dNTP (Cosmo gene tech), 50 mM Tris-HCl (pH 8.3), 75 mM KCl, 3 mM MgCl₂, 8 mM DTT, and 200 units of MMLV reverse transcription polymerase (Invitrogen), further adding DW treated with DEPC to be a final volume of 20 μl, mixing, and then reacting the synthesizing reaction solution at 25° C. for 10 minutes, at 37° C. for 50 minutes, and then at 70° C. for 15 minutes in a thermocycler (ABI).

b. Performance of Realtime PCR

For a composition of a realtime PCR reactant, 25 mM TAPS (pH 9.3 at 25° C.), 50 mM KCl, 2 mM MgCl₂, 1 mM 2-mercaptoethanol, 200 μM each dNTP, 1 unit of a Taq polymerase (TAKARA), 10 pmole of a Forward primer, 10 pmole of a Reverse primer, 10 pmole of a probe, and 2 μl of the synthesized cDNA were added, and a realtime PCR was carried out to be a final volume of 20 μl. The primers and probes had the following base sequences, respectively.

Primer and Probe for CK19

Forward:
(Sequence No. 13)
5′GATGAGCAGGTCCGAGGTTA-3
Reverse:
(Sequence No. 14)
5′TCTTCCAAGGCAGCTTTCAT-3
Probe:
(Sequence No. 15)
5′FAM-CTGCGGCGCACCCTTCAGGGTCT-BHQ1-3

Primer and Probe for EpCAM

Forward:
(Sequence No. 16)
5′GCCAGTGTACTTCAGTTGGTGCAC-3
Reverse:
(Sequence No. 17)
5′CATTTCTGCCTTCATCACCAAACA-3
Probe:
(Sequence No. 18)
5′FAM-TACTGTCATTTGCTCAAAGCTGGCTGCCA-BHQ1-3

Primer and Probe for hTERT

Forward:
(Sequence No. 19)
5′TGACGTCCAGACTCCGCTTCAT-3
Reverse:
(Sequence No. 20)
5′ACGTTCTGGCTCCCACGACGTA-3
Probe:
(Sequence No. 21)
5′FAM-GCTGCGGCCGATTGTGAACATGGA-BHQ1-3

Primer and Probe for Ki67

Forward:
(Sequence No. 22)
5′TAATGAGAGTGAGGGAATACCTTTG-3
Reverse:
(Sequence No. 23)
5′AGGCAAGTTTTCATCAAATAGTTCA-3
Probe:
(Sequence No. 24)
5′FAM-GGCGTGTGTCCTTTGGTGGGCA-BHQ1-3

Primer and Probe for Vimentin

Forward:
(Sequence No. 25)
5′ATGTTGACAATGCGTCTCTGGCA-3
Reverse:
(Sequence No. 26)
5′ATT TCCTCTTCGTGGAGTTTCTTCAAA-3
Probe:
(Sequence No. 27)
5′FAM-TGACCTTGAACGCAAAGTGGAATCTTTGC-BHQ1-3

The PCR was carried out by using an ABI 7500Fast (Applied Biosystem) one time at a denaturation temperature of 94° C. for 5 minutes; and a cycle of a denaturation temperature of 95° C. for 30 seconds and an annealing temperature of 55° C. for 20 seconds was repeatedly carried out 40 times. In addition, a fluorescence measurement process was added after each of the annealing processes to measure a fluorescent value that was increased in each cycle.

(5) Result Analysis

Each of experiment results was analyzed by using 7500 Software v2.0.4 (Applied Biosystem). EpCAM and CK19 as surface antigens were classified as positive or negative based on a Ct value from the RT-qPCR. When Ct values of the two markers were equal to or less than 38, they were determined as positive, and when the Ct values were equal to or more than 38, they were determined as negative. As for each of the markers, expression amounts from the patient group were comparatively quantitated based on an expression amount of HER2, hTERT, and Ki67 expressed in the blood of the normal persons. In this case, each of marker expression amounts was compared based on an expression amount of GAPDH.

1. RT-qPCR Used in Biopsy for Drug Screening

1-1) Comparison of HER2 Expression Amount for Each Cell Line Using RT-qPCR

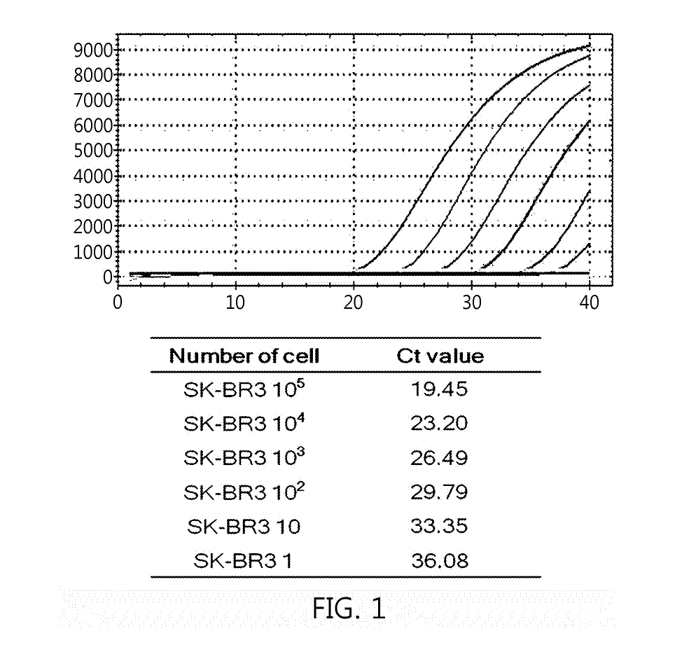

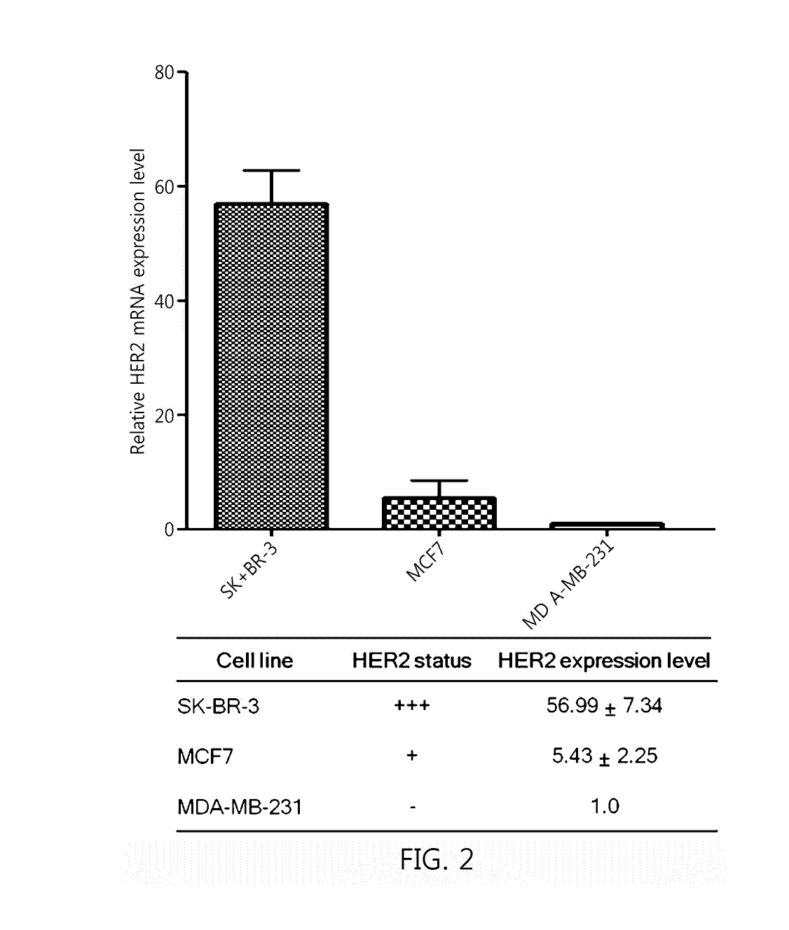

SK-BR-3 as HER2 positive breast cancer cell line was used to check HER2 sensitivity. As a result thereof, it can be seen from FIG. 1 that HER2 sensitivity made it possible to detect even a single SK-BR-3 cell by using an RT-qPCR, Further, SK-BR3, MCF7, and MDA-MB-231 cell lines as breast cancer cell lines were used to compare HER2 expression amounts with each other. When an expression amount of MDA-MB-231 cell line as a HER2 negative cell line was set to 1 as a reference value, it could be seen that an HER2 expression amount of MCF7 was presented as about 5.4 and an HER2 expression amount of SK-BR-3 was presented as about 56.9.

1-2) Setting of Clinical Cut-Off

An HER2 RT-qPCR was carried out by using FFPE specimens from 199 breast cancer patients in the Shinchon Severance Hospital from, and then, a result thereof was compared with IHC scores and FISH results of the breast cancer patients. After the IHC scores were set to be scored 0 for IHC 0, 25 for IHC 1+, 50 for IHC 2+ and FISH negative, 75 for IHC 2+ and FISH positive, and 100 for IHC 3+, a correlation analysis was conducted with a result thereof and the result of the HER2 RT-qPCR.

Referring to FIG. 3, Pearson r was 0.5418, R square was 0.2936, and P value was <0.0001. Thus, it could be seen that there was a correlation between the RT-qPCR result and the IHC result.

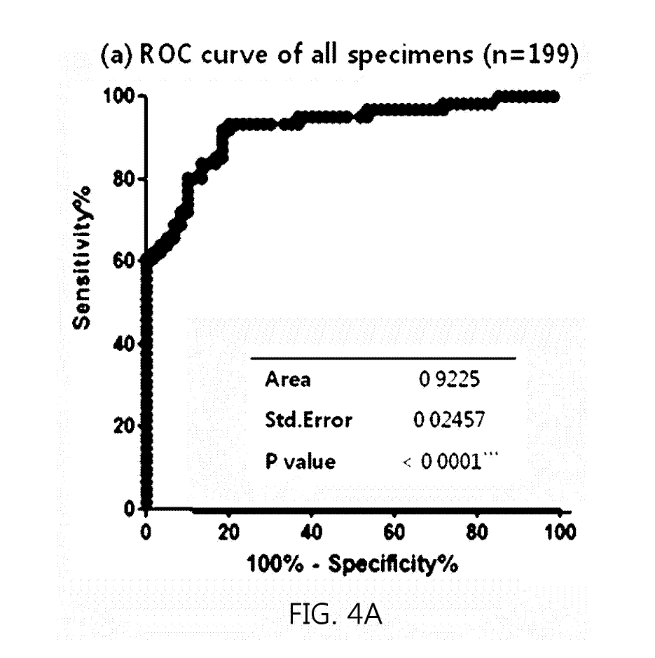

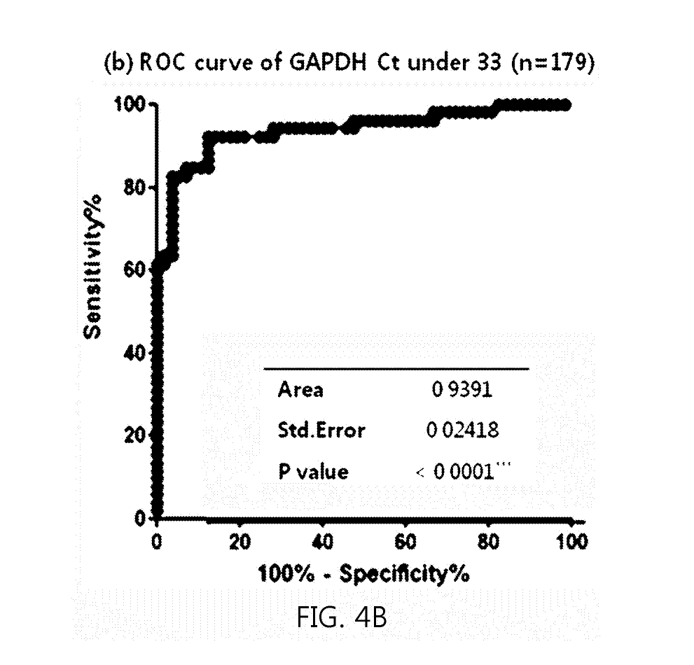

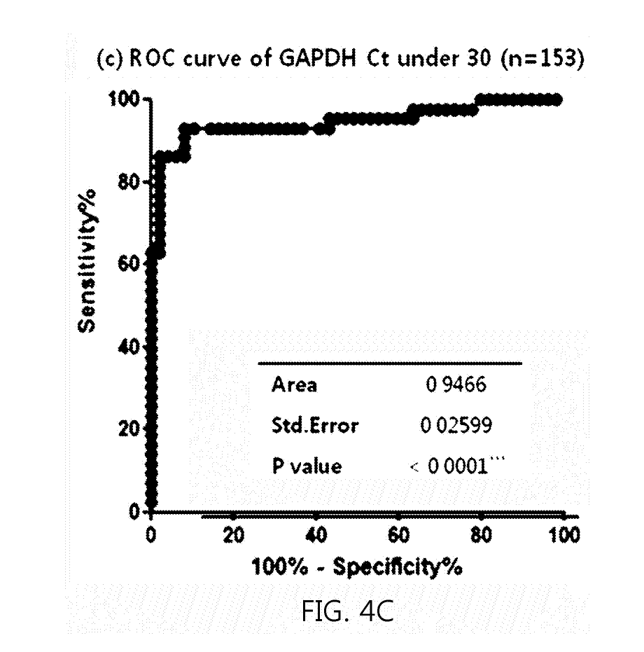

Further, a RNA quality is very important in an experiment using an FFPE specimen. A specimen having a high RNA quality can produce an accurate result, whereas a specimen having a low RNA quality can produce a false positive or false negative result. Therefore, in the present invention, a RNA quality was presented based on an expression amount of GAPDH. As can be seen from FIG. 4A-C, according to an expression level of GAPDH classified based on a Ct value of the RT-qPCR, when GAPDH had a lower Ct value, a more accurate result could be produced.

Herein, an ROC (Receiver Operating Characteristic) curve is a graph that shows performance of a judgement result (binary classifier) of a certain test, and has a TPR (True Positive Rate) or sensitivity as the Y-axis and a FPR (False Positive Rate) or 1-specificity as the X-axis.

That is, a calculation was made as follows:

TRP=Y-axis=sensitivity=(TP/(TP+FN)

FPR=X-axis=1−specificity=1−[TN/(TN+FP)]

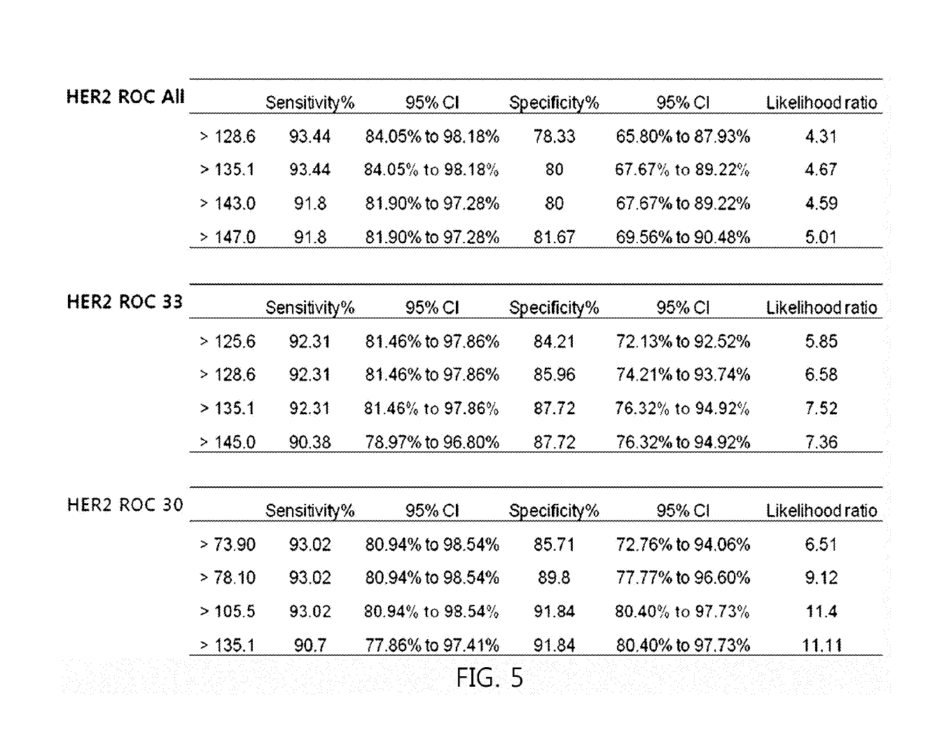

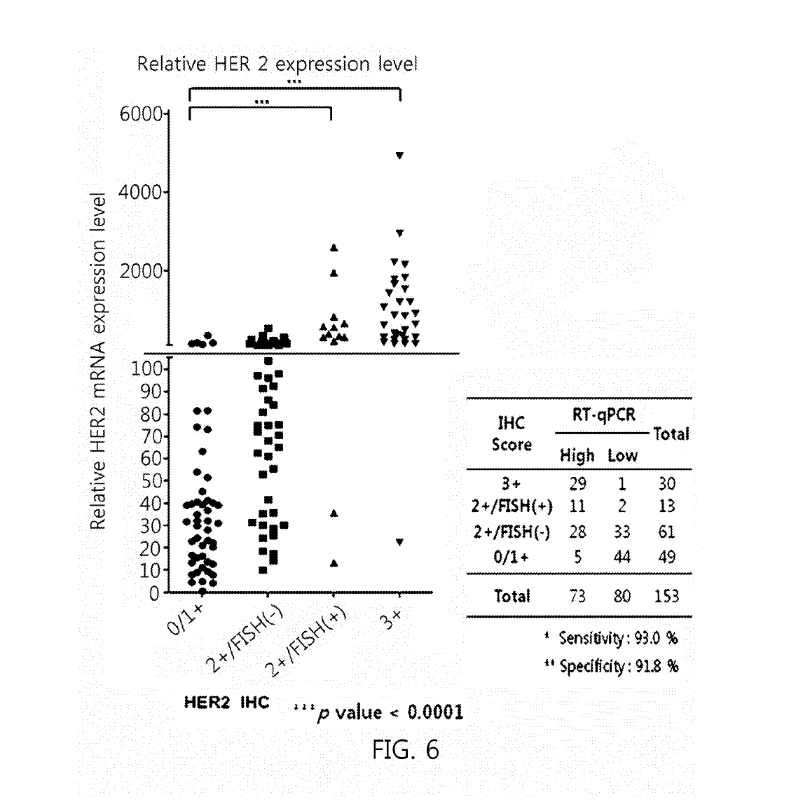

Referring to FIG. 5, it could be seen that when GAPDH had a Ct value of equal to or less than 30, its sensitivity and specificity was the highest (sensitivity: 93.02, specificity: 91.84), and in this case, a Cut-Off was 105.5. A result of analyzing the clinical specimens using a Cut-Off set as such was as shown in FIG. 6.

As can be seen from FIG. 6, specimens with IHC 0/1+ were analyzed as negative controls, and a specimen with IHC 2+/FISH positive and a specimen with IHC 3+ were analyzed as positive controls, and a result thereof was calculated. A specimen with IHC 2+/FISH negative remains clinically controversial and thus was excluded from a result calculation. According to N Engl J Med. 2008 Mar. 27; 358 (13):1409-11, it was proved that when patients with IHC 2+/FISH negative were administered with Herceptin as a HER2-targeted agent, it was effective. Therefore, the patients could not be diagnosed as HER2 positive or negative. Accordingly, except for that, sensitivity and specificity of the present invention was confirmed as 93.0% and 91.8%, respectively.

2. Analysis of HER2 Expression Marker and Cancer Expression Marker in Blood

2-1) Comparison in Sensitivity and Specificity

In order to check whether or not expression of HER2 could be detected in blood, in the present invention, SK-BR-3 as a breast cancer cell line overexpressing HER2 was mixed with blood of a normal person without breast cancer. Then, a RNA was extracted therefrom to check whether or not a cancer cell could be detected in blood.

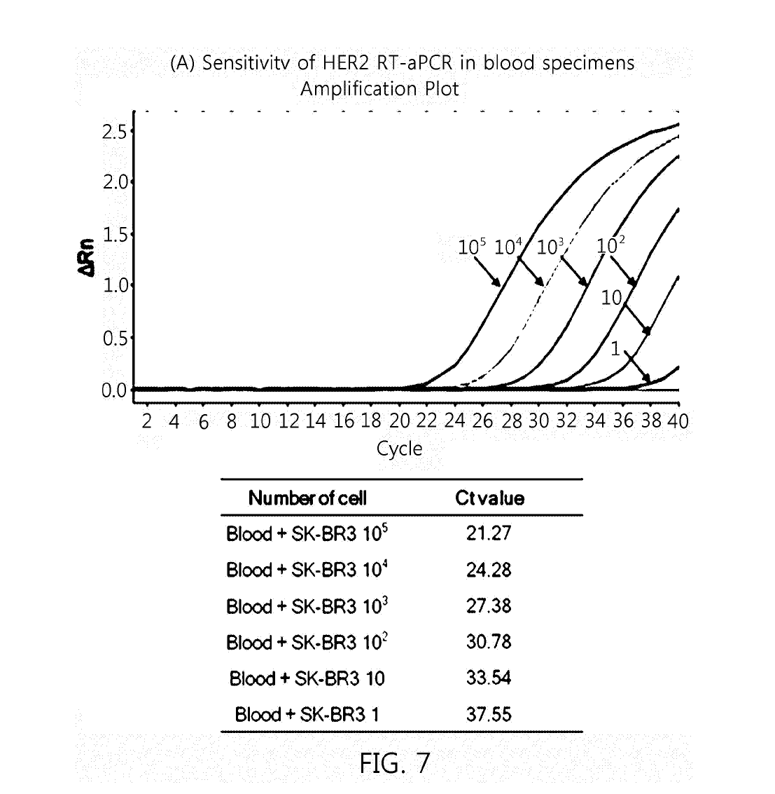

As can be seen from FIG. 7, HER2 sensitivity was so high as to make it possible to detect even a single SK-BR-3 cell mixed with the blood.

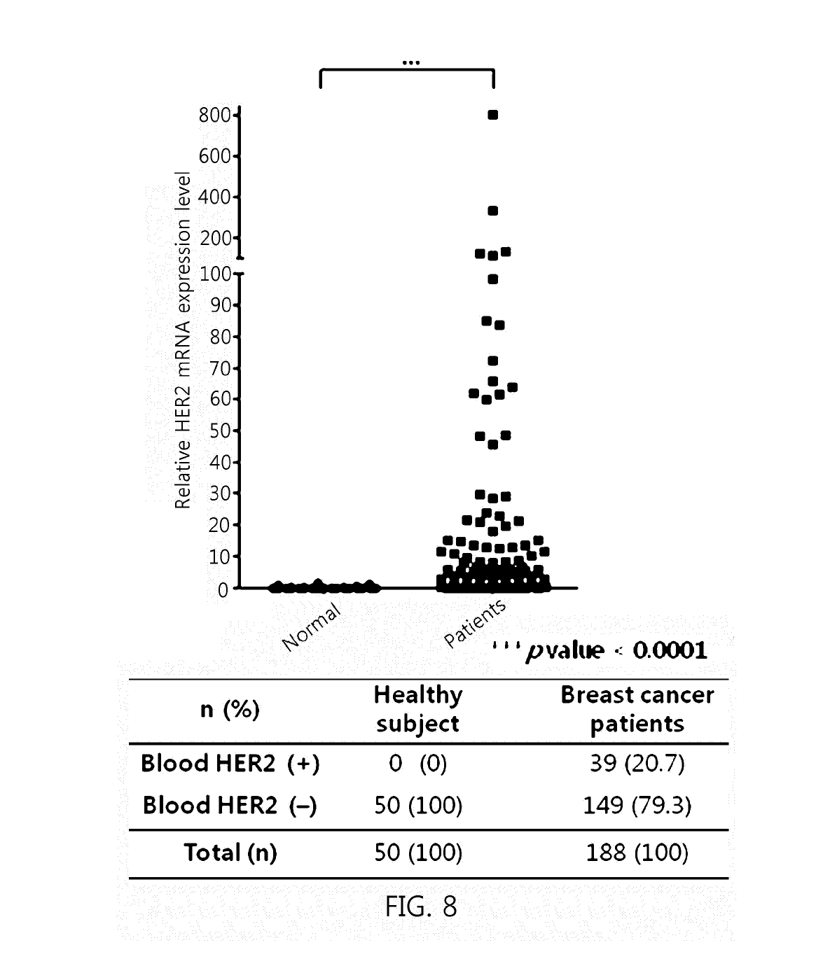

Further, in order to check whether or not there was expression of HER2 in blood, expression of HER2 was checked by using blood of 50 normal persons and blood of 188 breast cancer patients offered by the Shinchon Severance Hospital. As a result thereof, it could be found that expression levels s of HER2 in the normal persons were as low as 0 to 1.5, whereas various expressions levels of HER2 in the breast cancer patients could be seen in a range of 0 to 355. Among them, the patients having an HER2 expression level of 10 or higher were determined as positive, and an HER2 expression level equal to or higher than that was classified as positive and an HER2 expression level equal to or lower than that was classified as negative. A result thereof can be seen from FIG. 8.

As can be seen from FIG. 8, HER2 overexpression was not shown in the normal persons, whereas HER2 overexpression was not shown in 39 (20.7%) of the 188 breast cancer patients.

2-2) Comparison with Expression Amount of Cancer Marker in Other Blood

In order to check whether or not HER2 was actually expressed in blood by a cancer cell, whether or not there was expression of other markers related to the cancer cell was checked. The markers were EpCAM and Cytokeratin 19 as epithelial antigen markers and hTERT and Ki67 as cancer-related markers in a cell and were used to check whether or not there was a cancer cell in blood. SK-BR-3 was used to check sensitivity of the EpCAM and the CK19, and MDA-MB-231 was used to check sensitivity of the hTERT. Further, MCF7 was used to check sensitivity of the Ki67.

Vimentin is an intermediate filament protein normally related to fibroblasts or cells of mesenchyme origin such as hematopoietic cells and is not present in most normal epithelial cells. It is known that expression of vimentin in protoplasm is widely distributed and often exhibits distinctive perinuclear and subplasmalemmal accentuation. Presence of vimentin shows a property of epithelial cells which can independently survive. Therefore, it has been regarded that whether or not vimentin and cytokeratins are expressed can be used as an important marker in determining an aggressive character and a metastatic ability of breast cancer. Thus, in order to check whether or not there was expression of a marker, MDA-MB-231 was used to check sensitivity.

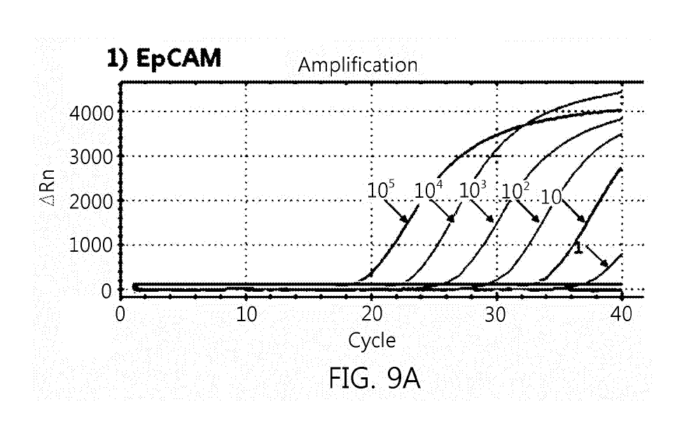

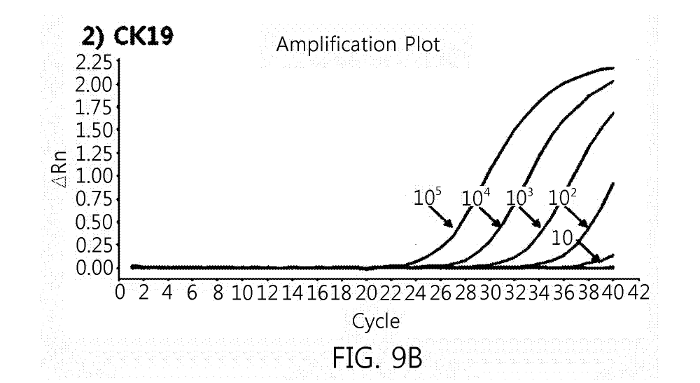

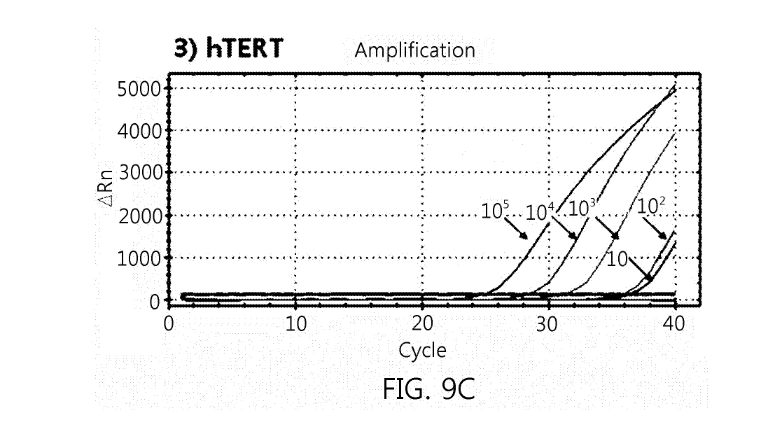

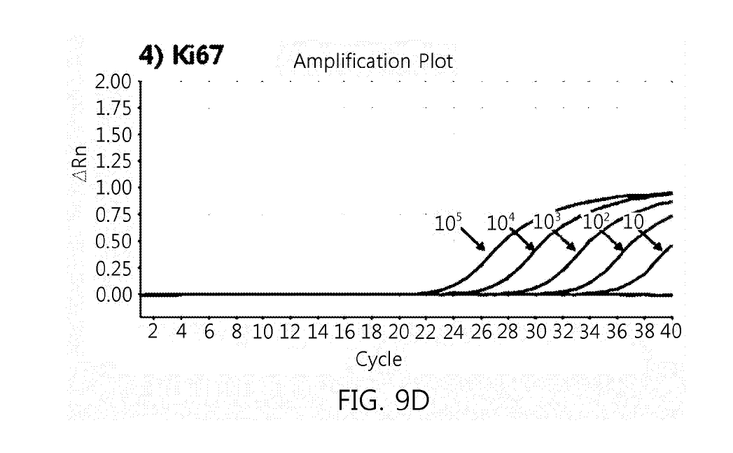

As can be seen from FIG. 9A-D, the EpCAM had sensitivity that made it possible to detect even a single cell in blood, and the CK19, the hTERT, and the Ki67 had sensitivity that made it possible to detect 10 cells in blood.

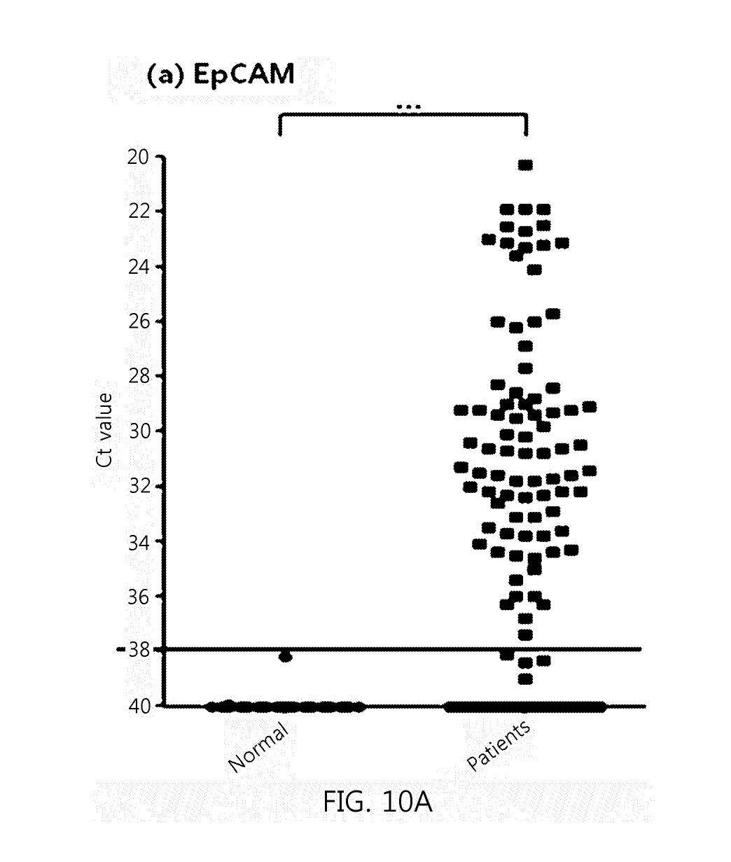

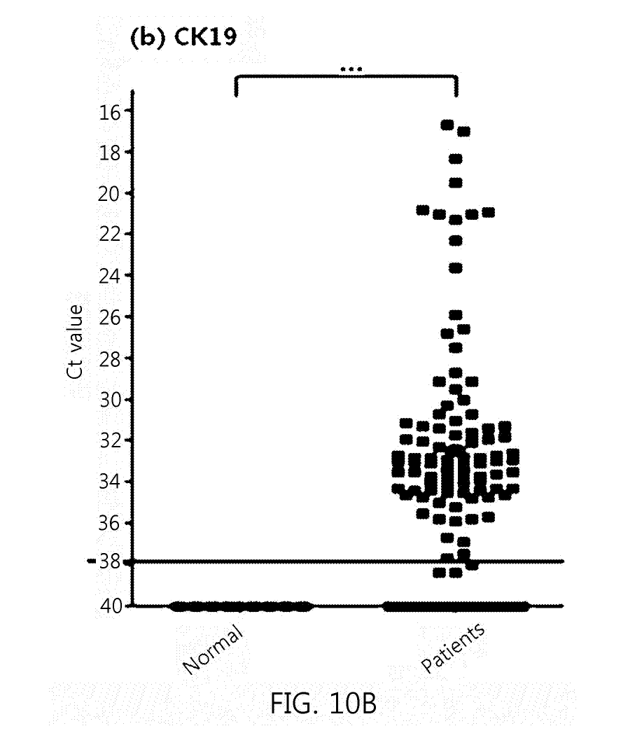

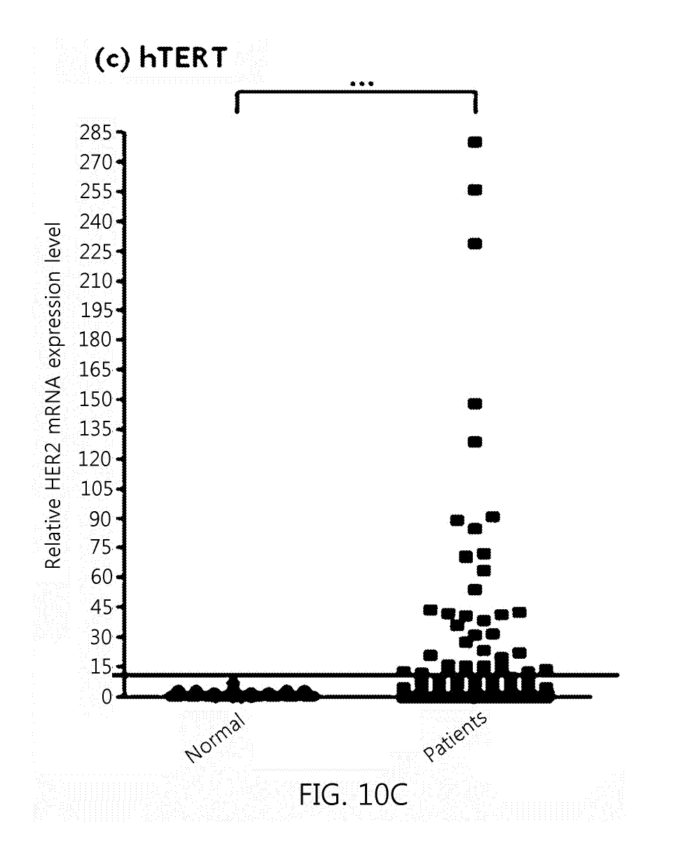

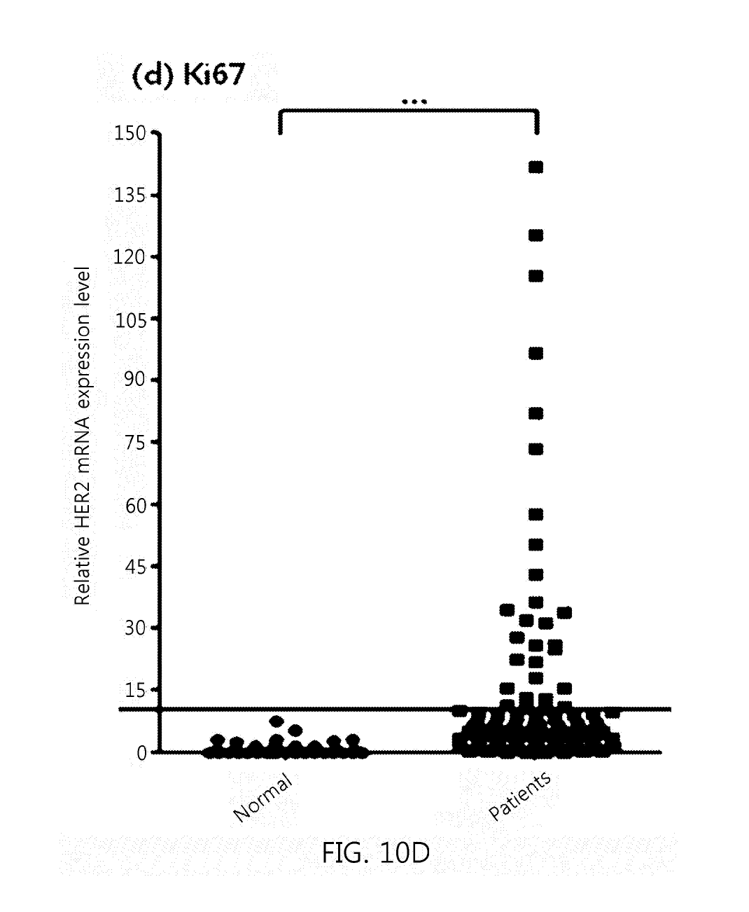

Further, as can be seen from FIG. 10A-D, a normal person and a breast cancer patient were compared in expression of the EpCAM and the CK19 by using a Ct value, and a normal person and the patient group were compared in a relative expression ratio between the hTERT the Ki67 in blood.

As can be seen from FIG. 10A-D, as for the EpCAM and the CK19, all of the normal persons except one had a low value in the EpCAM corresponding to a Ct value of 39 or higher, whereas some patients of the breast cancer patient group had a high value corresponding to a Ct value of 38 or lower. It could be seen that a positive rate of 45.2% was shown in the EpCAM and a positive rate of 50.5% was shown in the CK19. Further, as a result of measuring expression amounts of the hTERT and the Ki67, there was no normal person whose relative expression amounts of the hTERT and the Ki67 was 10 or higher. However, 20.7% of the breast cancer patient group had a high hTERT expression rate of 10 or higher and 13.8% of the breast cancer patient group had a high Ki67 expression rate of 10 or higher.

2-3) Comparison Between Expression Levels of HER2 and Breast Cancer-Related Marker in Blood

There was checked a relation between expression levels of a specimen of a patient who expressed HER2 in blood and the cancer-related marker used in the present invention.

As can be seen from FIG. 11, all of the patients who expressed HER2 in blood with the exception of two patients expressed an epithelial antigen or cancer-related marker in a cell at the same time. This confirmed that high expression of HER2 in blood was caused by presence of a cancer cell actually overexpressing HER2.

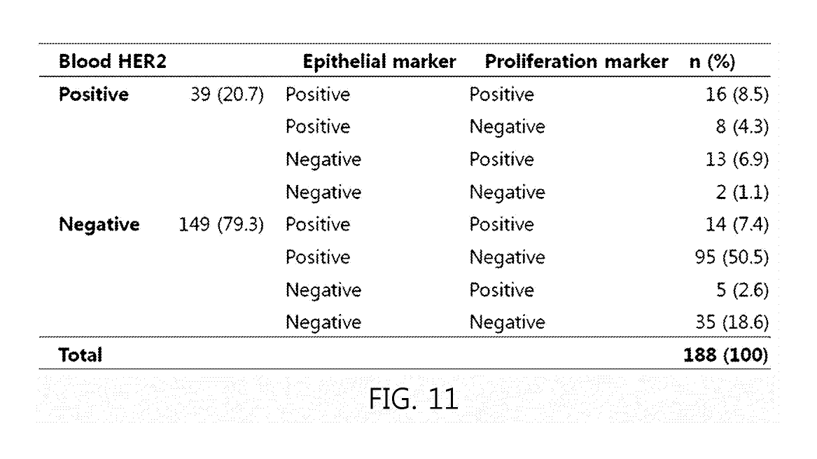

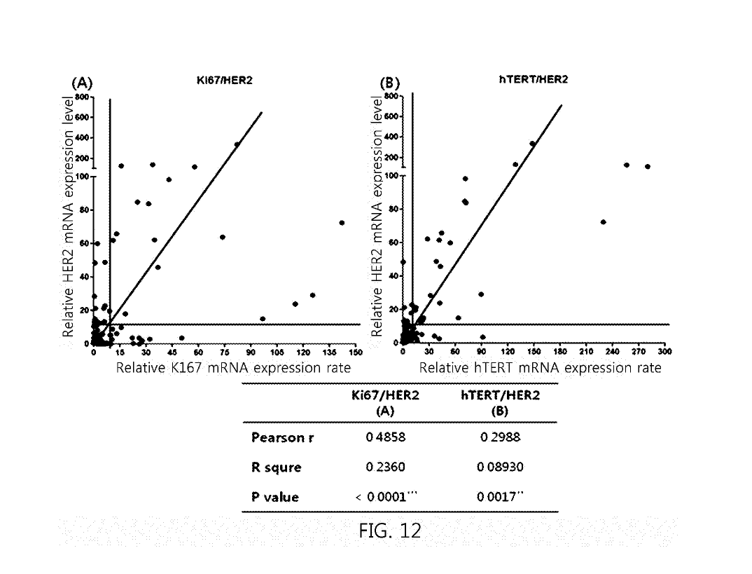

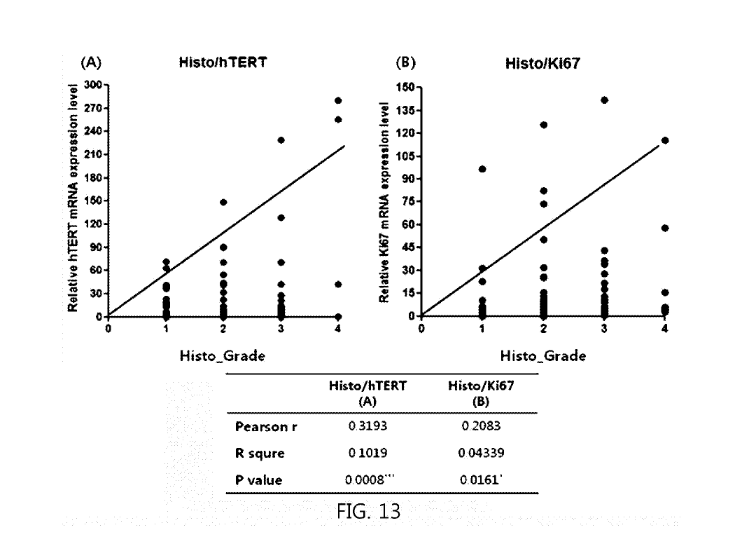

In particular, it could be seen that there was a correlation between expression of HER2 in blood and expressions of Ki67and hTERT in blood (FIG. 12). Therefore, it could be seen that overexpression of HER2 was involved in malignancy of a cancer cell, and also, it can be seen from FIG. 13 that along with gradual deterioration in a histological grade of a cancer cell, expressions of Ki67 and hTERT were involved to a certain extent.

2-4) Comparison Between Expression of HER2 in Blood and Result of Histological Examination on HER2

RT-qPCR was used to check a correlation between expression levels of HER2 expressed in blood and HER2 undergoing a histological examination by comparison.

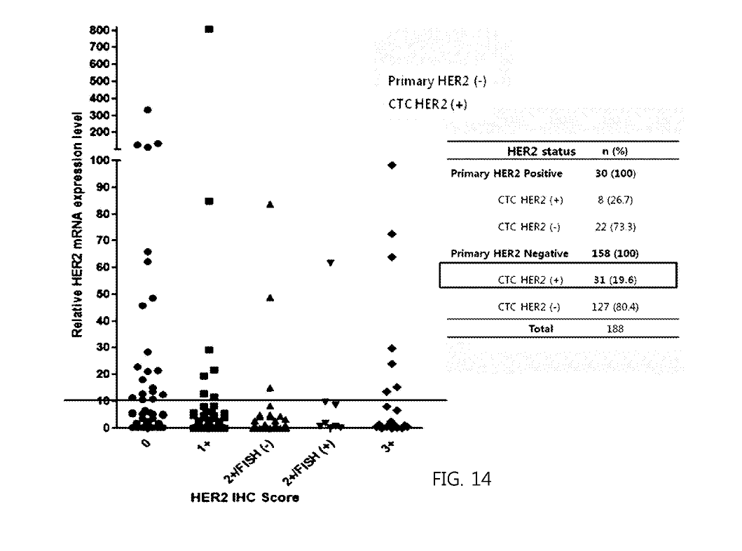

As can be seen from FIG. 12, there was a big difference between expression of HER2 in blood and a result of a histological examination on HER2. Referring to a table below in FIG. 14, a portion outlined in red square shows patients with HER2 negative as a result of a histological examination together with a high expression of HER2 in blood. Such patients account for as high as 19.6% of patients with HER2 negative, i.e. about one-fifth of the total. It could be seen that such patients were involved in malignancy of a cancer cell (FIG. 13). Therefore, it is expected that patients with high expression of HER2 in blood can be treated by administering Herceptin as a HER2-targeted agent to them.

2-5) Analysis of Cancer Marker in Blood by Stage of Breast Cancer

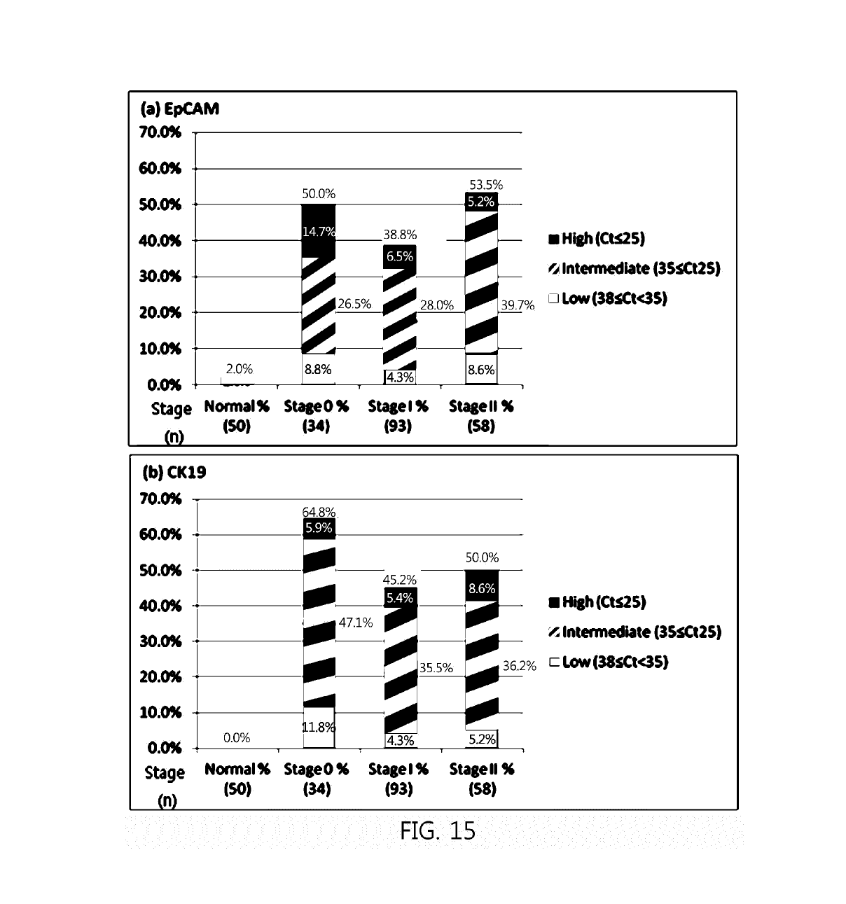

Analysis was conducted with a cancer marker in blood by stage of breast cancer. It can be seen from FIG. 15 that there was not much change in an epithelial antigen by stage of breast cancer. However, it could be seen that the epithelial antigen had a high expression level at Stage 0. It is deemed that this can be involved in early detection of breast cancer.

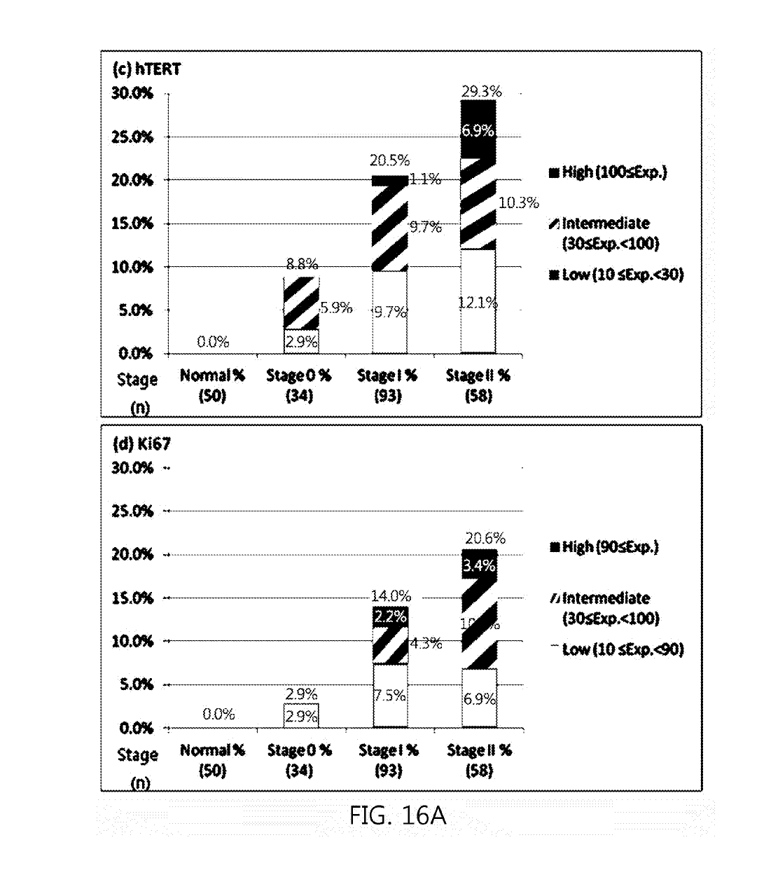

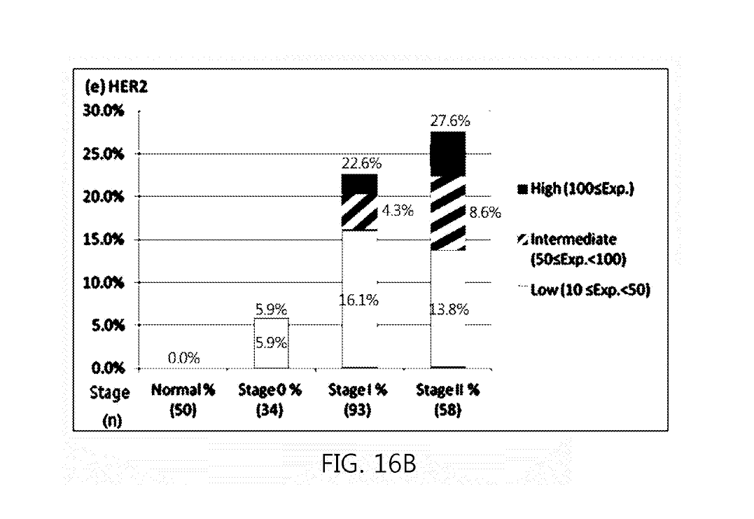

It can be seen from FIG. 16A-B that there was a change in expression level of a cancer-related marker in a cell by stage of breast cancer. It could be seen that as for all of hTERT and Ki67 as well as HER2, along with progression of the stage of breast cancer, expression of a cancer marker in blood is increased. In particular, it could be seen that a ratio of patients with overexpression as much as 90 to 100 times was increased along with progression of the stage of breast cancer. Therefore, this confirmed that a cancer-related marker in a cell is involved in stage of a patient. Thus, it is deemed that malignancy of breast cancer of a patient can be checked by means of a rapid diagnosis using blood.

2-6) Comparison of HER2 Expression Amount and Ct Value of RT-qPCR Using Single Primer Pair and Probe Versus Multiplex One-Tube Nested PCR

All protocol and condition are same in Example 1.

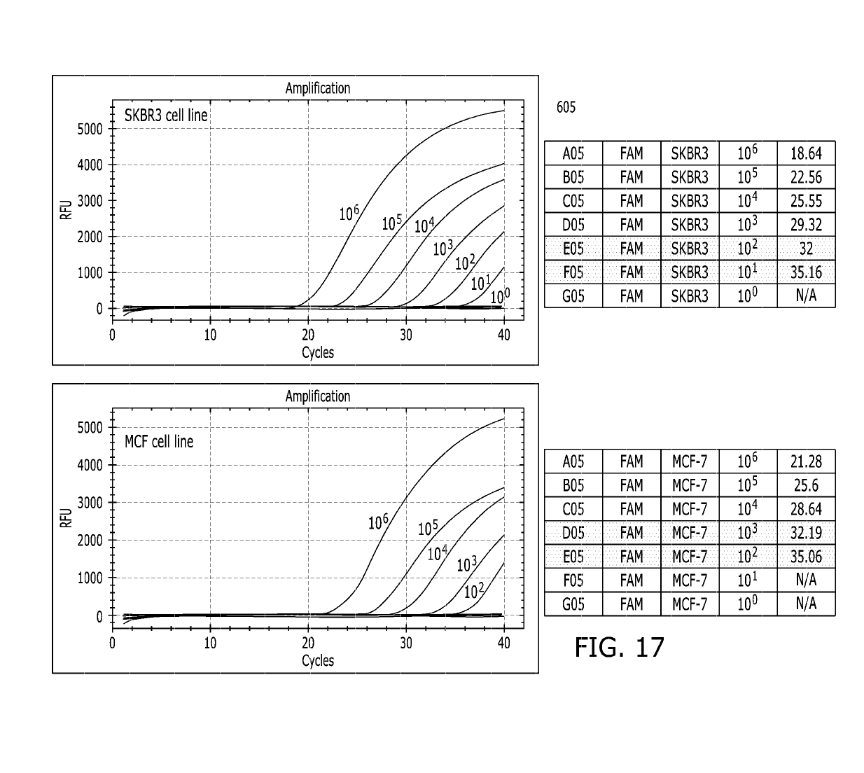

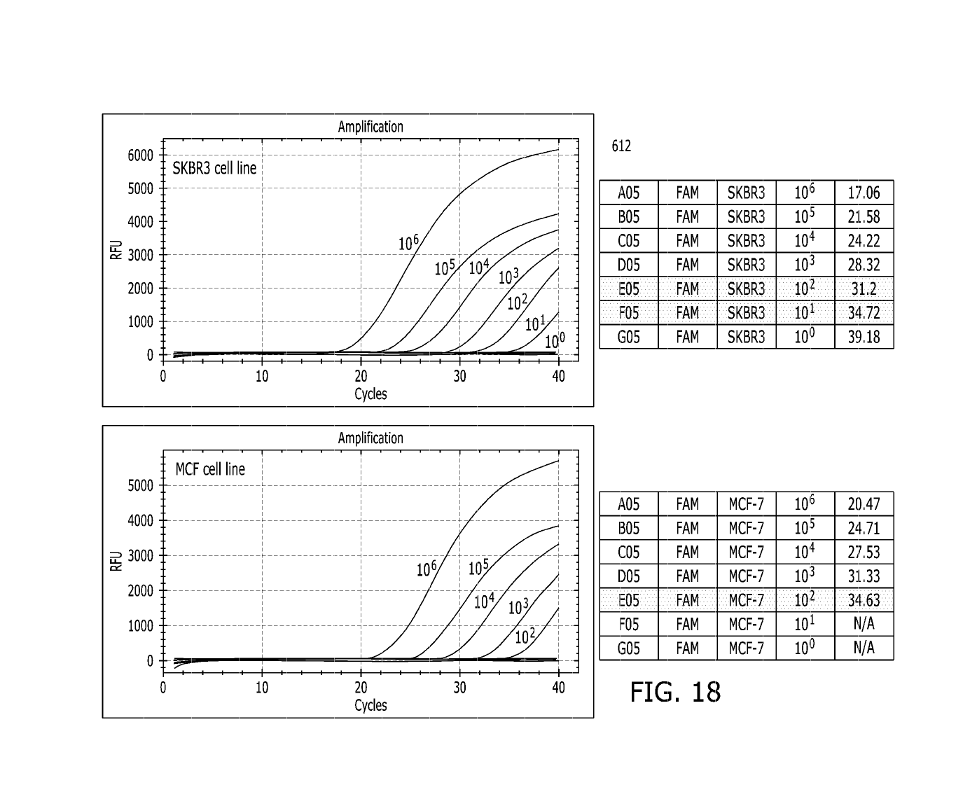

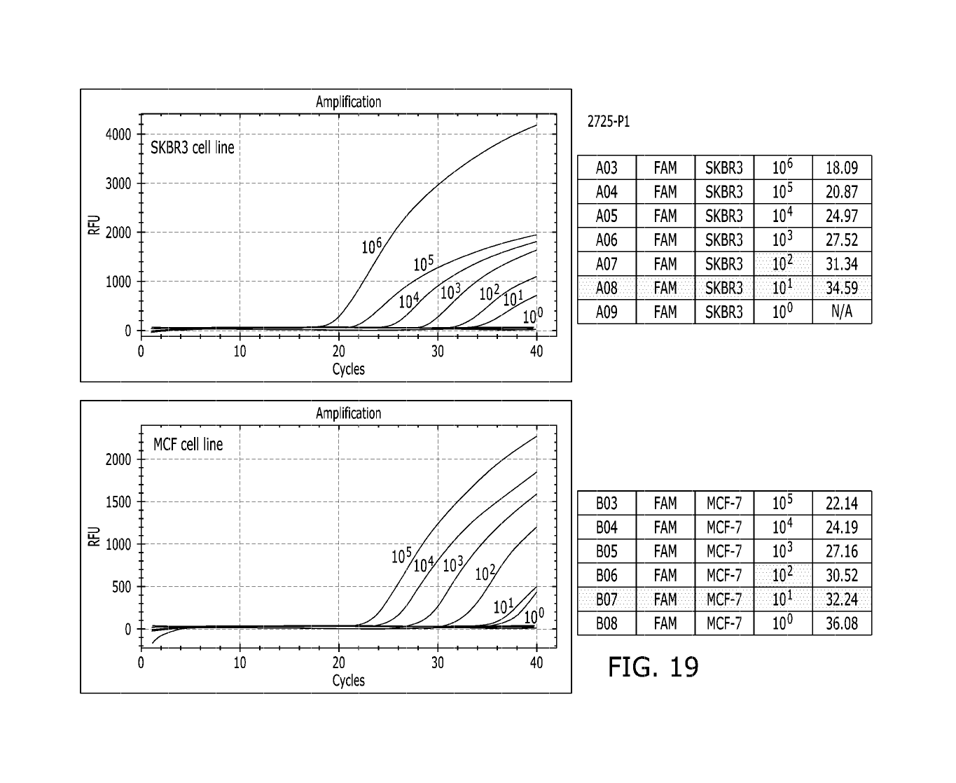

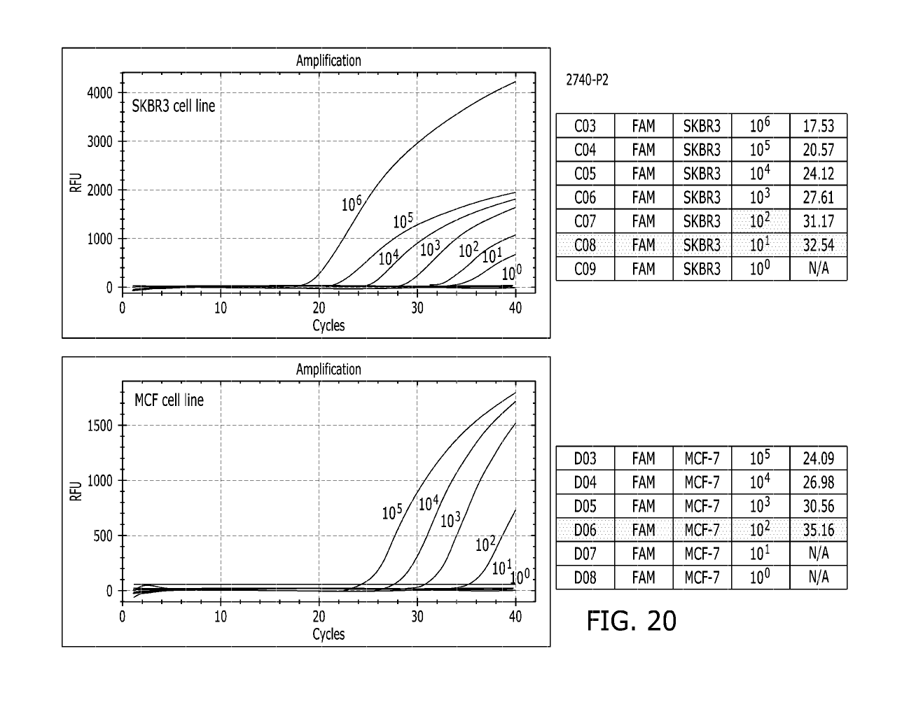

FIGS. 17-20 are the result of an RT-qPCR using single primer pair & probe (SEQ.ID No.1-2, and 5 in FIG. 17; SEQ.ID No.3-4, and 5 in FIG. 18; SEQ.ID No.6-7, and 8 in FIG. 19; or SEQ.ID No.6-7, and 9 in FIG. 20, respectively);

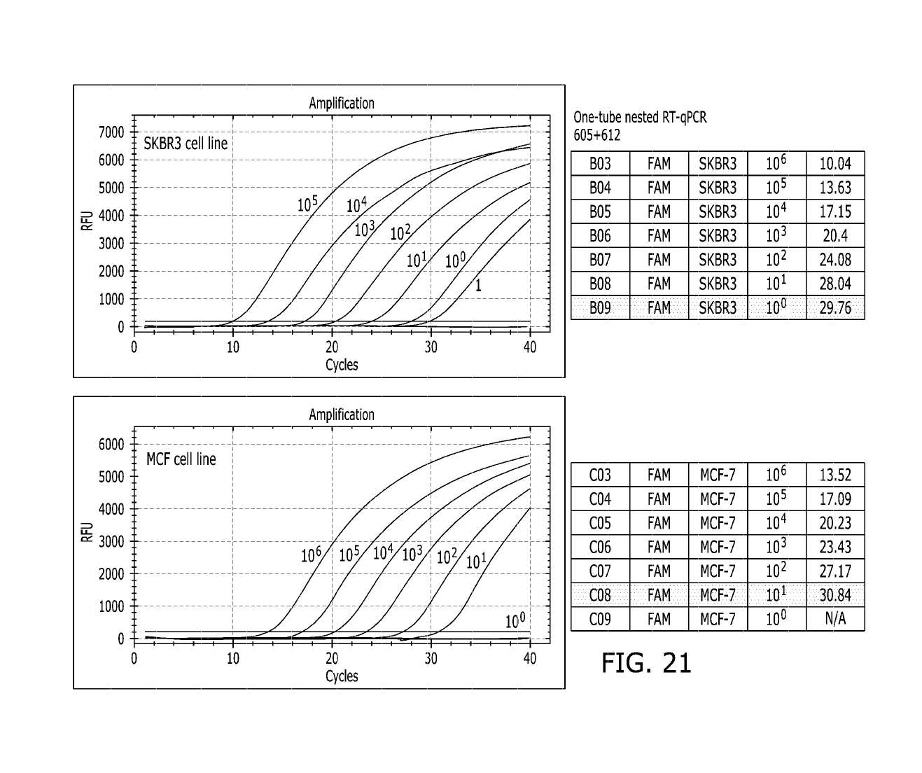

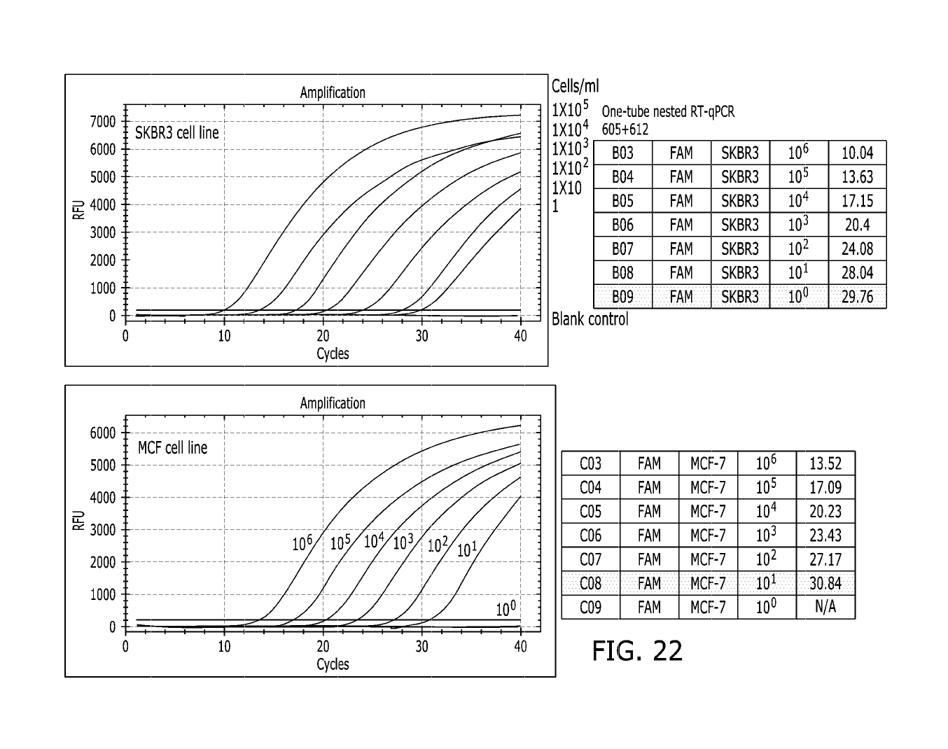

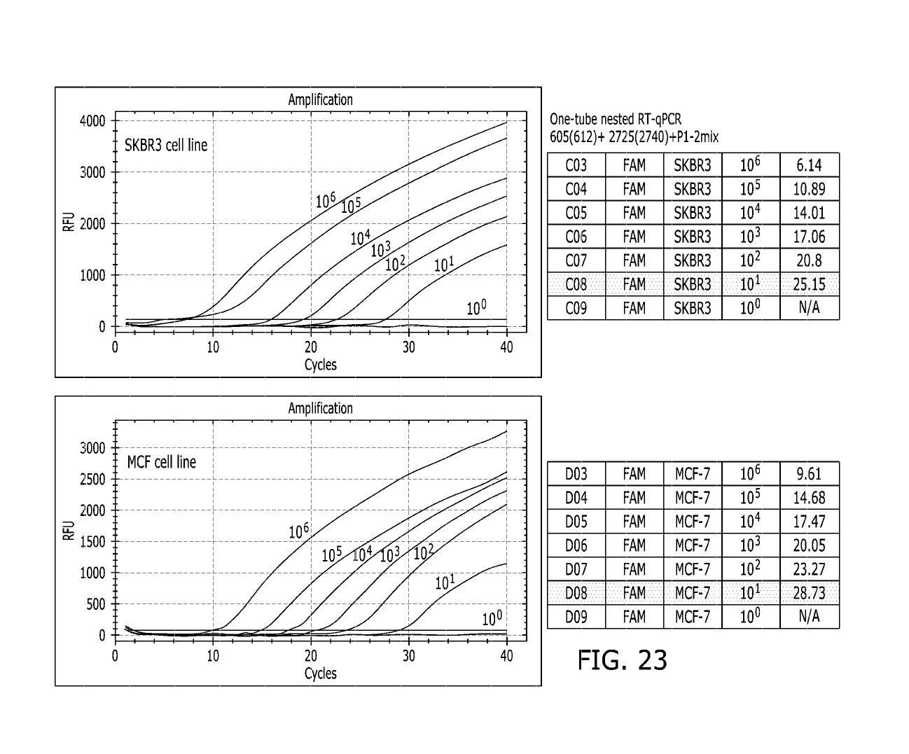

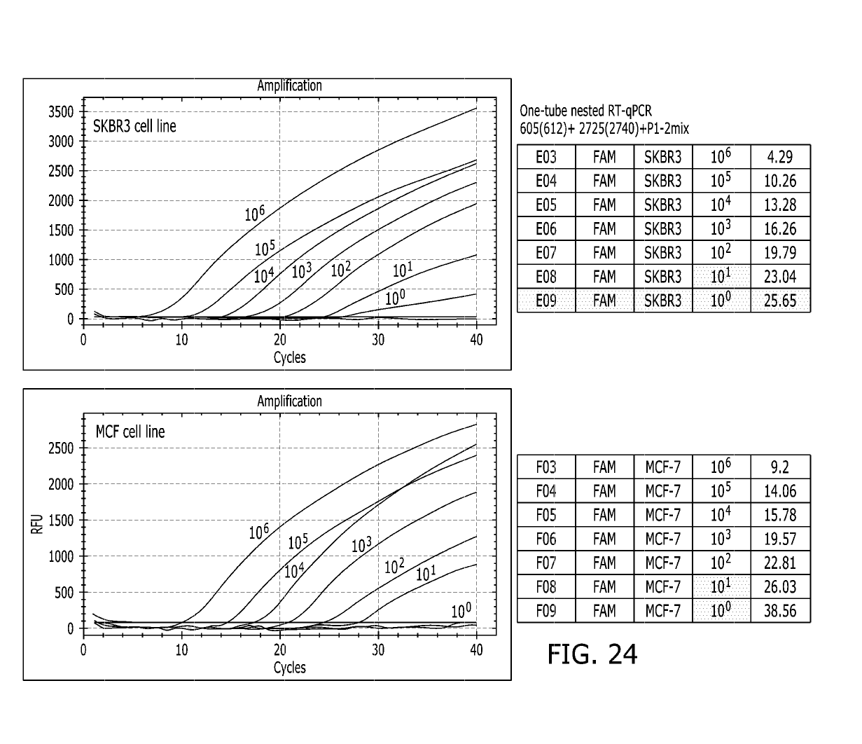

FIGS. 21-24 are the multiplex one-tube nested PCR result of an RT-qPCR using primer pairs & probes (SEQ.ID No.1-4, and 5 in FIGS. 21-22; SEQ.ID No.1-9 in FIGS. 23-24, respectively), specially, in FIGS. 23-24, all primers and probes set forth in SEQ. ID 1-9 are used in the PCR.

The results of FIGS. 17-20 show 17-24 of Ct value in 10⁶ cell line, while the multiplex and one-tube PCR result of FIGS. 21-24 show 4-10 of Ct value in that concentration, confirming enhanced HER2 sensitivity. Since 2-3 difference of Ct value means more than 100-fold of sensitivity, the above results suggest that HER2 sensitivity enhances more than 10¹⁰ -fold by the multiplex and one-tube PCR with the primer and probe mix of the present invention.

In addition, the results of FIGS. 17-20 using single primer and probe show more than 30 of Ct value in 10³-10² cell line, and so have difficulty in detecting in the cell line concentration, but the results of multiplex-one tube nested PCR using all primers and probes mix of the present invention show sensitivity which it is possible to detect even in 10¹-10⁰ cell line concentration.

1. An information offering method for diagnosing breast cancer comprising: (a) separating total RNA from a cell obtained from a tissue or blood of a suspected cancer patient; (b) synthesizing a cDNA from the separated total RNA; (c) performing a real-time PCR with the synthesized cDNA by using primer pair mix and probe mix that can amplify human epidermal growth factor receptor 2 (HER2); wherein, the primer pair mix that can amplify human epidermal growth factor receptor 2 (HER2) are described as SEQ ID Nos.:1 and 2, 3 and 4, and 6 and 7 and the probe mix are described as SEQ ID Nos.: 5, 8, and 9.


SEQUENCE LISTING (27 sequences)

SEQ ID NO: 1 - DNA, 26 bp, Artificial Sequence
  oligonucleotide primer
aacctggaactcacctacctgcccac

SEQ ID NO: 2 - DNA, 22 bp, Artificial Sequence
  oligonucleotide primer
cgatgagcacgtagccctgcac

SEQ ID NO: 3 - DNA, 23 bp, Artificial Sequence
  Oligonucleotide primer
aactcacctacctgcccaccaat

SEQ ID NO: 4 - DNA, 20 bp, Artificial Sequence
  oligonucleotide primer
cacgtagccctgcacctcct

SEQ ID NO: 5 - DNA, 25 bp, Artificial Sequence
  oligonucleotide probe
cagcctgtccttcctgcaggatatc

SEQ ID NO: 6 - DNA, 22 bp, Artificial Sequence
  oligonucleotide primer
aagcatacgtgatggctggtgt

SEQ ID NO: 7 - DNA, 24 bp, Artificial Sequence
  oligonucleotide primer
tctaagaggcagccatagggcata

SEQ ID NO: 8 - DNA, 26 bp, Artificial Sequence
  Oligonucleotide probe
atatgtctcccgccttctgggcatct

SEQ ID NO: 9 - DNA, 25 bp, Artificial Sequence
  oligonucleotide probe
catccacggtgcagctggtgacaca

SEQ ID NO: 10 - DNA, 22 bp, Artificial Sequence
  oligonucleotide primer
ccatcttccaggagcgagatcc

SEQ ID NO: 11 - DNA, 21 bp, Artificial Sequence
  oligonucleotide primer
atggtggtgaagacgccagtg

SEQ ID NO: 12 - DNA, 24 bp, Artificial Sequence
  oligonucleotide probe
tccacgacgtactcagcgccagca

SEQ ID NO: 13 - DNA, 20 bp, Artificial Sequence
  oligonucleotide primer
gatgagcaggtccgaggtta

SEQ ID NO: 14 - DNA, 20 bp, Artificial Sequence
  oligonucleotide primer
tcttccaaggcagctttcat

SEQ ID NO: 15 - DNA, 23 bp, Artificial Sequence
  oligonucleotide probe
ctgcggcgcacccttcagggtct

SEQ ID NO: 16 - DNA, 24 bp, Artificial Sequence
  oligonucleotide primer
gccagtgtacttcagttggtgcac

SEQ ID NO: 17 - DNA, 24 bp, Artificial Sequence
  oligonucleotide primer
catttctgccttcatcaccaaaca

SEQ ID NO: 18 - DNA, 29 bp, Artificial Sequence
  oligonucleotide probe
tactgtcatttgctcaaagctggctgcca

SEQ ID NO: 19 - DNA, 22 bp, Artificial Sequence
  oligonucleotide primer
tgacgtccagactccgcttcat

SEQ ID NO: 20 - DNA, 22 bp, Artificial Sequence
  oligonucleotide primer
acgttctggctcccacgacgta

SEQ ID NO: 21 - DNA, 24 bp, Artificial Sequence
  oligonucleotide probe
gctgcggccgattgtgaacatgga

SEQ ID NO: 22 - DNA, 25 bp, Artificial Sequence
  oligonucleotide primer
taatgagagtgagggaatacctttg

SEQ ID NO: 23 - DNA, 25 bp, Artificial Sequence
  oligonucleotide primer
aggcaagttttcatcaaatagttca

SEQ ID NO: 24 - DNA, 22 bp, Artificial Sequence
  oligonucleotide probe
ggcgtgtgtcctttggtgggca

SEQ ID NO: 25 - DNA, 23 bp, Artificial Sequence
  oligonucleotide primer
atgttgacaatgcgtctctggca

SEQ ID NO: 26 - DNA, 27 bp, Artificial Sequence
  oligonucleotide primer
atttcctcttcgtggagtttcttcaaa

SEQ ID NO: 27 - DNA, 29 bp, Artificial Sequence
  oligonucleotide probe
tgaccttgaacgcaaagtggaatctttgc